(19) United States
(12) Patent Application Publication
(10) Pub. No.: US 2024/0386629 A1
(43) Pub. Date: Nov. 21, 2024

(54) INTRA-IMAGE NONRIGID MOTION CORRECTION IN TOMOGRAPHY

(71) Applicant: California Institute of Technology, Pasadena, CA (US)

(72) Inventors: Peng Hu, Alhambra, CA (US); Lihong Wang, Arcadia, CA (US)

(21) Appl. No.: 18/658,435

(22) Filed: May 8, 2024

Related U.S. Application Data
(60) Provisional application No. 63/464,832, filed on May 8, 2023.
(60) Provisional application No. 63/464,812, filed on May 8, 2023.

(51) Int. Cl. G06T 11/00; G06T 7/20

(52) U.S. Cl. CPC G06T 11/008; G06T 7/20

(57) ABSTRACT

Certain aspects pertain to intra-image motion correction methods and systems configured to divide an image domain into subdomains and estimate motion of each subdomain along one or more axes and determine a motion corrected image based on the motions.

### CROSS-REFERENCES TO RELATED APPLICATIONS

This application claims priority to and benefit of U.S. Provisional Patent Application No. 63/464,812, titled “A Hybrid Method For Fast Functional Imaging With Sparse Sampling In Tomography,” filed on May 8, 2023 and U.S. Provisional Patent Application No. 63/464,832, titled “Intra-Image Nonrigid Motion Correction In Tomography,” filed on May 8, 2023; both of which are hereby incorporated by reference in their entireties and for all purposes.

### FEDERALLY SPONSORED RESEARCH OR DEVELOPMENT

This invention was made with government support under Grant No(s). NS102213 & EB029823 & CA220436 awarded by the National Institutes of Health. The government has certain rights in the invention.

### FIELD

Certain aspects generally pertain to tomography, and more specifically to, methods for intra-image motion correction in tomography.

### BACKGROUND

Tomographic imaging modalities x-ray computed tomography (CT), magnetic resonance imaging (MRI), and photoacoustic computed tomography (PACT) produce cross-sectional images of tissue by detection of penetrating x-rays, nuclear-magnetic-resonance-induced radio waves, and light absorption-induced ultrasonic waves, respectively. Each modality with a certain setup is described by a system matrix. Tomographic imaging modalities are generally described by large system matrices. Tissue motion can degrade the system matrix and image quality. For example, tissue motions such as heart beating, breathing, abdominal movement, and fetal movement, can cause complex geometric errors in each system matrix, which introduce artifacts in the reconstructed image and compromise valuable image features.

Various methods have been proposed to compensate for system-matrix imperfections from image-domain, signal-domain, and cross-domain perspectives. However, due to the large size of each system matrix, these methods tend not to manipulate or correct the system matrix directly and have limitations. For example, for intra-image nonrigid motion correction, gating- and binning-based methods are commonly used. However, these methods require repeated data acquisition, which is time-consuming and infeasible for unrepeated motions. Deep neural networks (DNNs) have also been used for motion correction, however, DNNs need specific training datasets that are not universally available, and it is challenging to reject falsely generated features in DNNs.

Two system-matrix-level methods have been proposed for motion correction. A first method approximates general nonrigid motions with localized linear translations, identifies possible motion paths from multichannel navigator data, and estimates the motion at each pixel using localized gradient-entropy metric in the image domain. However, quantifying localized motion from only navigator data is not robust, especially when the motion amplitude and noise level increase. A second method expresses breathing- and heartbeat-induced motions in basis functions by performing singular value decomposition (SVD) and resolves these motions in imaging. However, for general motions, especially unrepeated motions, the method's performance is unknown. The high computation cost of SVD in the method also restricts its application to 3D imaging.

### SUMMARY

Techniques disclosed herein may be practiced with a processor-implemented method, a system comprising one or more processors and one or more processor-readable media, and/or one or more non-transitory processor-readable media.

Certain embodiments pertain to intra-image motion correction methods. In some cases, an intra-image motion correction method includes dividing an image domain into a plurality of subdomains and reconstructing a tomographic image. The method also includes estimating a motion of a subdomain along a first axis and reconstructing a first motion-corrected image using the motion of the subdomain along the first axis. In one case, the method also estimates a motion of the subdomain along a second axis and estimates a motion of the subdomain along a third axis, wherein the motion corrected image is determined using the motion of the subdomain along the first axis, the motion of the subdomain along the second axis, and the motion of the subdomain along the third axis.

Certain embodiments pertain to a system including a tomographic imaging device comprising one or more sensor arrays, one or more processors in electrical communication with the tomographic imaging device, and one or more processor-readable media. The one or more processor-readable media store instructions which, when executed by the one or more processors, cause performance of (a) dividing image domain into a plurality of subdomains, (b) reconstructing a tomographic image, (c) estimating a motion of a subdomain along a first axis; and (d) reconstructing a first motion-corrected image using the motion of the subdomain along the first axis. In one cases, (c) further comprises (c1) estimating a motion of the subdomain along a second axis and (c2) estimating a motion of the subdomain along a third axis, wherein the motion corrected image is determined using the motion of the subdomain along the first axis, the motion of the subdomain along the second axis, and the motion of the subdomain along the third axis.

These and other features are described in more detail below with reference to the associated drawings.

### BRIEF DESCRIPTION OF THE DRAWINGS

FIG. 1A depicts an illustration of a virtual transducer hemispherical array formed by rotation of arc arrays of a PACT system and exemplary responses of three virtual sensor elements to signals from three voxels, according to an embodiment.

FIG. 1B depicts an illustration of piece-wise compression of a tomographic imaging system matrix compression (TISMC) method, according to an embodiment.

FIG. 2A is an illustration depicting the forward simulation procedure of a TISMC method that simulates sensor signals, according to embodiments.

FIG. 2B depicts an illustration of system matrix slicing in voxel indices and virtual sensor element indices, according to an embodiment.

FIG. 3 depicts a flowchart illustrating operations of a TISMC method, according to various embodiments.

FIG. 4 depicts a flowchart illustrating operations of an intra-image nonrigid motion correction method, according to various embodiments.

FIG. 5A depicts a MAP of a human breast image, according to an embodiment.

FIG. 5B depicts a schematic drawing of intra-image motions induced by tissue translation, according to an embodiment.

FIG. 5C depicts a schematic drawing of intra-image motions induced by tissue deformation, according to an embodiment.

FIG. 6A depicts MAPs of the images reconstructed from the signals with motions induced by tissue translation without and with motion correction, according to an embodiment.

FIG. 6B depicts MAPs of the images reconstructed from the signals with motions induced by tissue deformation without and with motion correction, according to an embodiment.

FIG. 7A depicts expanded views of subsets of the MAPs enclosed in dashed boxes in FIG. 6A and ground truth image for comparison, according to an embodiment.

FIG. 7B depicts expanded views of subsets of the MAPs enclosed in dotted boxes in FIG. 6B and ground truth image for comparison, according to an embodiment.

FIG. 8A depicts MAPs of a human breast image reconstructed without and with motion correction, according to an embodiment.

FIG. 8B depicts MAPs of a human breast image reconstructed without and with motion correction, according to an embodiment.

FIG. 9A depicts an illustration of a transducer element, a single point source, and a transducer element local coordinate system with axes \hat{x}ₙ, ŷₙ, and \hat{z}ₙ, according to an embodiment.

FIG. 9B depicts an illustration of four point sources in the local coordinate system of the transducer element in FIG. 9A, according to an embodiment.

FIG. 10A depicts a graph of four responses of the transducer element in FIG. 9B to the signals from the four point sources, according to an embodiment.

FIG. 10B depicts an illustration of four responses to the signals from four point sources in FIG. 9B using linear combinations of three temporal singular functions based on a singular value decomposition (SVD) procedure, according to an embodiment.

FIG. 11A depicts illustrations of responses of transducer element to fifty (50) point sources with decreasing distances, the temporally-shifted form, white noise responses, according to an embodiment.

FIG. 11B depicts a graph with normalized singular values in the SVDs of the signals, according to an embodiment.

FIG. 11C depicts a graph with proportions of the variances in the SVDs of the signals, according to an embodiment.

FIG. 12A depicts an illustration of a numerical phantom formed by three rectangular cuboids intersecting at their centers, according to an embodiment.

FIG. 12B depicts a virtual 2D array, four subdomains, and the domain occupied by rotations of the four subdomains around the array axis, according to an implementation.

FIG. 12C depicts a plot of computation times of the fast and slow operators for the forward simulations, according to an implementation.

FIG. 13A depicts images of relative errors of the simulated signals for all virtual elements with the numerical phantom in four subdomains, according to an implementation.

FIG. 13B depicts a comparison of the signals simulated by the fast and slow operators, according to an implementation.

FIG. 14 are plots of the relative error of a reconstructed image in four subdomains with 1 to 256 iterations, values on three lines in the reconstructed images with 1, 4, 8, 64, and 256 iterations, and a comparison between ground truth and the 256-iteration reconstructed values along the three lines, according to an implementation.

FIG. 15 depicts a flowchart of a workflow of an example of a motion correction method, according to an embodiment.

FIG. 16A depicts an illustration of signals from the first human breast in vivo experiment, according to an embodiment.

FIG. 16B depicts an illustration of signals from the second human breast in vivo experiment, according to an embodiment.

FIG. 17A depicts an illustration of signals from human breast imaging in vivo, and images reconstructed with and without motion correction, according to an embodiment.

FIG. 17B depicts an illustration of signals from human breast imaging in vivo, and images reconstructed with and without motion correction, according to an embodiment.

FIG. 18A depicts a block diagram of a tomographic imaging system having a computing system, according to various embodiments.

FIG. 18B depicts a block diagram of an example of components of a computing system, according to an embodiment.

These and other features are described in more detail below with reference to the associated drawings.

### DETAILED DESCRIPTION

Different aspects are described below with reference to the accompanying drawings. The features illustrated in the drawings may not be to scale. In the following description, numerous specific details are set forth in order to provide a thorough understanding of the presented embodiments. The disclosed embodiments may be practiced without one or more of these specific details. In other instances, well-known operations have not been described in detail to avoid unnecessarily obscuring the disclosed embodiments. While the disclosed embodiments will be described in conjunction with the specific embodiments, it will be understood that it is not intended to limit the disclosed embodiments.

### I. Introduction

Certain embodiments pertain to methods for intra-image nonrigid motion correction (also referred to herein as “intra-image motion correction methods” or “motion correction methods”) that can correct for motion within a tomographic image that is induced by tissue translation and deformation. These motion correction methods approximate the motion within the tomographic image with localized linear translations. Starting from an initially reconstructed motion-blurred image, the translation of each subdomain of an object being imaged is estimated by minimizing the difference between the simulated signals from the subdomain and the detected signals. With the estimated translations, the tomographic system matrix is updated (corrected) and the tomographic image is reconstructed again. The correction-reconstruction process is iterated to obtain a final tomographic image. These motion correction methods do not require repeated data acquisition and are effective for unrepeated motions (compared to time-gating-based methods). The motion correction methods are robust in revealing true structures and avoiding false structures (compared to DNN-based methods).

In one implementation, a motion correction method also includes a process for compressing the tomographic system matrix. For example, the motion correction method may first perform a compression method to compress a tomographic system matrix, which enables efficient system matrix slicing and manipulation. Enabled by this efficiency, one or more images may be corrected. An example of a suitable compression process that may be employed is an imaging system matrix compression (TISMC) method (also sometimes referred to herein as a “compression methods”). In some cases, TISMC methods temporally shift one or more of the responses to align arrival times and then group aligned responses for each virtual sensor element. The TISMC methods then perform a grouped SVD procedure (for each virtual sensor element) on the aligned responses to approximate the responses as linear combinations of a plurality of basis functions to obtain a compressed system matrix (piece-wise compression). For each combination of voxel and virtual sensor element, the coefficients for the basis functions are applied with the original arrival times for each virtual sensor element and convolutions are performed (e.g., implemented by FFT) to obtain simulated sensor signals for all virtual sensor elements. An iterative reconstruction procedure is applied to obtain a tomographic image from the detected signals. These TISMC methods improve computational efficiency (e.g., 42 times), which can enable efficient system matrix slicing and manipulation. This efficiency allows us to perform a larger number (e.g., 10000) of data-driven manipulations of subsets of the system matrix to improve the image quality, which is too time-consuming using traditional methods.

In both numerical simulations and human breast imaging in vivo discussed herein, motion correction methods of certain embodiments have been demonstrated to successfully mitigate motion-induced artifacts and recover motion-compromised features in tomographic images. Due to its low requirement in data acquisition and high robustness, motion correction methods described herein can be broadly applied to brain, heart, chest, abdominal, and fetus tomographic imaging. In certain implementations, motion correction methods can be used to reduce the effects of motions in a set of signals acquired in a single data acquisition run to form a single tomographic image. In another implementation, a motion correction method may be used to perform finer manipulations of the system matrix to recover a complete motion profile from these signals, which is an important feature for heart imaging. The recovered complete motion profile will be used to estimate if the heart is pumping normally and if there are any health issues in the heart.

### II. Methods

The intra-image motion correction methods described herein can be applied to various tomographic imaging techniques such as, e.g., a 3D photoacoustic computed tomography (3D PACT), ultrasound computed tomography (USCT), x-ray computed tomography (CT), and magnetic resonance imaging (MRI). Section IV discusses the application of intra-image motion correction methods to various tomographic imaging techniques in more detail. An example of a 3D PACT system is described in Lin, L. et al. “High-speed three-dimensional photoacoustic computed tomography for preclinical research and clinical translation,” Nat. Commun. Vol. 12, pp. 1-10 (2021), which is hereby incorporated by reference in its entirety and for all purposes. Another example of a 3D PACT system is described in U.S. patent application Ser. No. 16/798,204, titled “PHOTOACOUSTIC COMPUTED TOMOGRAPHY (PACT) SYSTEMS AND METHODS,” filed on Feb. 20, 2020, which is hereby incorporated by reference in its entirety and for all purposes.

In certain examples discussed herein, the 3D PACT system disclosed in Lin, L. et al. “High-speed three-dimensional photoacoustic computed tomography for preclinical research and clinical translation,” Nat. Commun. Vol. 12, pp. 1-10 (2021) is used to demonstrate examples of intra-image motion correction methods. In many of these examples, the PACT system includes a four 256-element 2-D arc ultrasonic transducer arrays where each 2-D arc array has a central frequency of 2.25 MHz and one-way bandwidth of 98%. The 3D PACT system is used to demonstrate intra-image motion correction methods due to its representatively large size in tomography: light-absorption-induced ultrasonic wave from every voxel in an image is detected by every transducer element and the system matrix is intrinsically a 6D tensor. In certain examples, intra-image motion correction methods are applied to both numerical simulations and in vivo experiments: intra-image motion correction in human breast imaging.

### A. Tomographic Imaging System Matrix Compression (TISMC) Methods

According to various embodiments, a TISMC method can compress a tomographic imaging system response matrix, which may advantageously enable efficient system matrix slicing and manipulation. The TISMC method temporally shifts one or more of the system responses to align arrival times and then groups the aligned responses for each virtual sensor element. The TISMC method then performs a grouped SVD procedure (for each virtual sensor element) to the aligned responses to approximate the responses as linear combinations of a plurality of basis functions (e.g., three temporal singular functions) to obtain coefficients for a compressed system matrix (piece-wise compression). In some cases, the TISMC method may also include forward simulations. For example, for each combination of voxel and virtual sensor element, the coefficients for the basis functions may be applied with the original arrival times for each virtual sensor element and convolutions (e.g., three convolutions) are performed (e.g., implemented by FFT) to obtain simulated sensor signals for all virtual sensor elements. This fast forward operator can be used in an iterative reconstruction procedure to obtain a tomographic image from the detected signals. TISMC methods improve computational efficiency (e.g., up to 42 times), which can enable efficient system matrix slicing and manipulation.

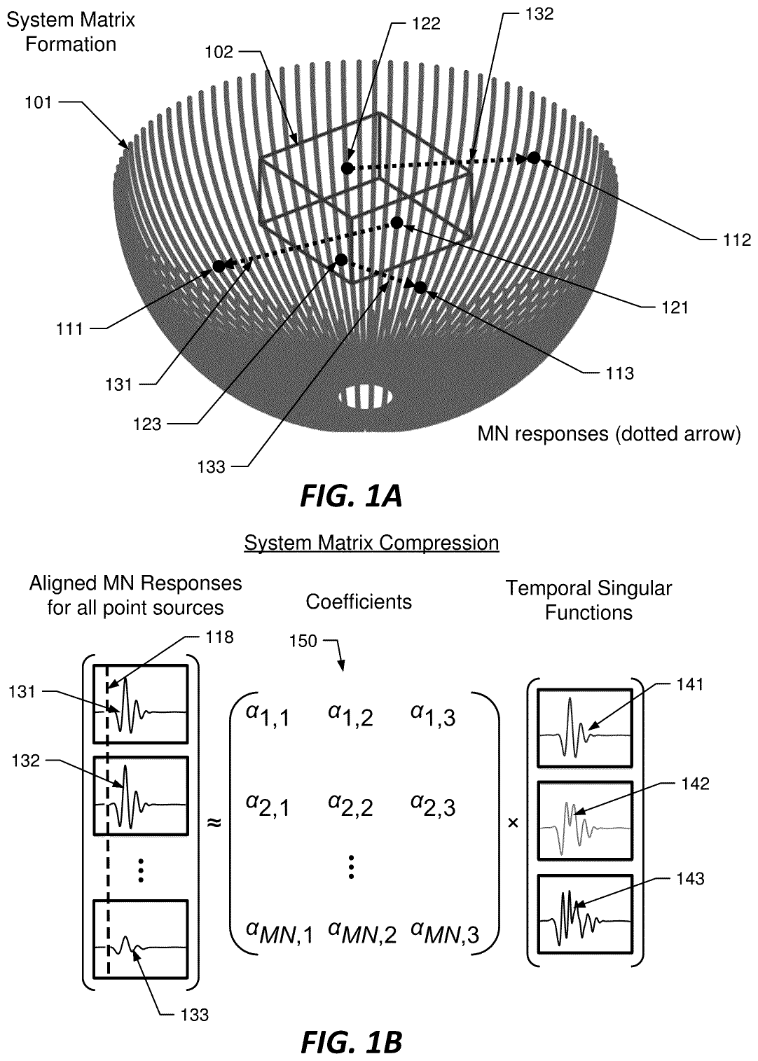

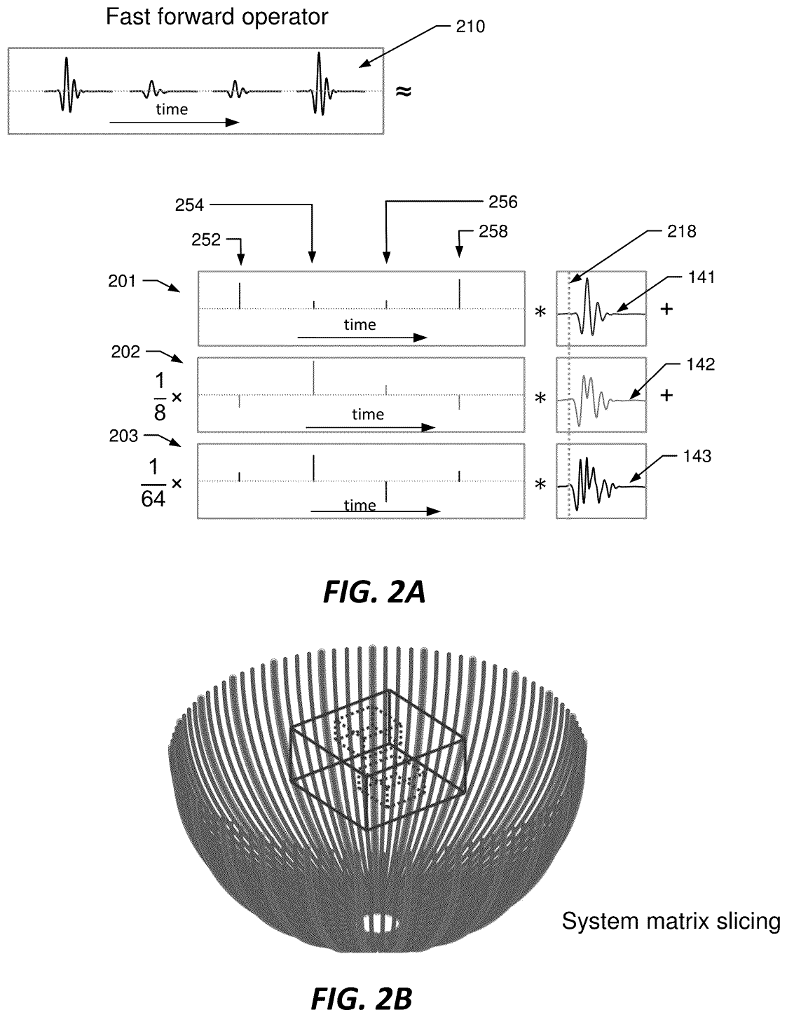

FIGS. 1A-1B and 2A-2B discuss an example of a TISMC method, according to an embodiment, that is used to compress a tomographic system matrix of the PACT system described in Lin, L. et al. “Highspeed three-dimensional photoacoustic computed tomography for preclinical research and clinical translation,” Nat. Commun. Vol. 12, pp. 1-10 (2021). The PACT system includes four 256-element arc ultrasonic transducer arrays where each arc array has a central frequency of 2.25 MHz and one-way bandwidth of 98%. The object being imaged was assumed to be a homogenous medium for simplicity. Although discussed with respect to a PACT system, it would be understood that the TISMC method also applies to other tomographic imaging systems such as CT and MRI systems.

FIG. 1A depicts an illustration of a virtual hemispherical array 101 formed by the rotation of the four 256-element arc transducer arrays in the PACT system. The illustration also shows an image domain with a 3D tomographic image 102 depicted as a rectangular cuboid with solid edges. The illustration also includes the paths of three system responses 131, 132, and 133 denoted by dotted arrows from three voxels 121, 122, and 123 (point sources) to three virtual transducer elements 111, 112, and 113. The virtual hemispherical array 101 is truncated at the bottom for light delivery. The detected ultrasonic signals from the four arc transducer arrays were used to form the 3D tomographic image 102.

The number of voxels in the 3-D image and the number of virtual sensor elements are denoted as M and N, respectively in various examples. During data acquisition, the physical sensors are rotated/translated while detecting signals at different locations. Virtual sensor elements are defined at the various detection locations of the physical sensors. A tomographic imaging system matrix is formed of MN responses of all N virtual sensor elements to the signals from all M voxels in the 3D tomographic image. For instance, in the example illustrated in FIG. 1A, the number of voxels in the 3D tomographic image 102 and the number of virtual transducer elements in the virtual hemispherical array are denoted as M and N, respectively. For illustration purposes, the paths of three responses 131, 132, and 133 are denoted by dotted arrows from three voxels (denoted as dots) 121, 122, and 123 to three virtual transducer elements (denoted as dots) 111, 112, and 113. Without compression techniques, the system matrix may be as large as, e.g., 600*600*300*400*256*151 non-zero elements It can be expensive to store and manipulate a system matrix with a large number of responses directly from memory and computation perspectives, respectively.

According to various embodiments, a TISMC method compresses the system matrix, which can advantageously enable efficient system matrix slicing and manipulation. First, considering that arrival times of the responses are determined by the voxel-element distances (i.e. distances between the voxel and the virtual transducer), TISMC methods of various embodiments may temporally shift one or more of the responses to align the arrival times to remove the differences in response arrival times. FIG. 1B depicts the three responses 131, 132, and 133 from FIG. 1A that are temporally shifted to align arrival times and the aligned responses grouped by sensor element are then compressed based on an SVD procedure, according to an embodiment. As shown, the first response 131, second response 132, . . . , MN response 133 have been temporally shifted to align along a dashed line 118 to remove the differences in arrival times from the voxels to elements, and the responses are compressed based on SVD with the first three temporal singular functions 141, 142, and 143, respectively. In other implementations, other basis functions may be used.

In various embodiments, a TISMC method temporally shifts one or more of the responses so that the arrival times are aligned to remove the differences in response arrival times and then groups the responses for each sensor element. The TISMC method then performs a grouped SVD procedure to the shifted responses to approximate the responses as linear combinations of a plurality of basis functions to obtain a compressed system matrix (piece-wise compression). The grouped SVD procedure obtains for each combination of source point and sensor element, a coefficient for each basis function that is used in the linear combinations. An example of basis functions that can be used are three temporal singular functions (e.g., three temporal singular functions 141, 142, and 143 shown in FIG. 1B).

For example, the TISMC method being implemented in FIG. 1B groups the responses for each virtual transducer element. The TISMC method then performs a grouped SVD procedure to the shifted responses to approximate the responses as linear combinations (where coefficients are shown in FIG. 1B) of the three temporal singular functions 141, 142, and 143 for efficient compression of the system matrix. Section III(A) has additional detail of the process of performing SVD to the responses grouped for each virtual element in the element's local coordinate system. Due to the homogeneous-medium assumption and identity of all elements in the system, SVDs for all virtual elements share the same set of temporal singular functions including the first three temporal singular functions 141, 142, and 143 shown in FIG. 1B. In other implementations, another plurality of basis functions may be used.

The system matrix compression of a TISMC method not only saves memory in storing the system matrix but also may enable fast and accurate forward simulation. Once the system matrix is compressed, the TISMC method can simulate the sensors signals using the plurality of coefficients obtained during piece-wise compression. The TISMC method determines a simulated signal at each virtual sensor element. For all the voxels, the TISMC method determines a time series of coefficients for each basis function based on the actual arrival times. After obtaining all the coefficients with different arrival times for each basis function for each virtual sensor element, the TISMC method performs a plurality of convolutions (e.g., implemented by FFT) on the time series of coefficients for the plurality of basis functions to obtain a simulated sensor signal for each virtual sensor. Based on the fast forward operator, an iterative reconstruction procedure can then be used to reconstruct one or more 3D images from the detected signals. An example of an iterative reconstruction procedure is the iterative gradient descent algorithm. Another example of a suitable iterative reconstruction procedure is the fast iterative shrinkage-thresholding algorithm FISTA, which can be found in Beck, A. & Teboulle, M. A, “fast iterative shrinkage-thresholding algorithm for linear inverse problems,” SIAM J. Imaging Sci. 2, 183-202 (2009), which is hereby incorporated by reference in its entirety. An example of a suitable iterative reconstruction procedure is the nonnegativity-constrained iterative method described by Eqn. 33.

FIG. 2A is an illustration depicting the forward simulation procedure of a TISMC method that simulates sensor signals, according to embodiments. In this example, for each combination of voxel and virtual sensor element, instead of adding the whole weighted response to the signals, only three coefficients are applied: values of the first three spatial singular functions 141, 142, and 143 from FIG. 1B.

For each of the voxels (point sources), the TISMC method determines a time series of coefficients for each basis function based on the original (unshifted) arrival times of the original system matrix. In the illustrated example, three temporal singular functions 141, 142, and 143 are being used and three time series of coefficients 201, 202, and 203 are obtained. The time series of coefficients 201, 202, and 203 include coefficients for a first point source (voxel) 252, coefficients for a second point source 254, coefficients for a third point source 256, and coefficients for a fourth point source 258. After obtaining all the coefficients with different arrival times for each virtual element, the TISMC method performs three convolutions (implemented by FFT) to the times series of coefficients to approximate the simulated signals. A compressed system matrix with the simulated signals using the original system matrix can then be formed. FIG. 2A includes a simulated signal 210 of a transducer element approximated by three convolutions with the three temporal singular functions 141, 142, and 143 where (zero) arrival time is indicated by a dotted line 218.

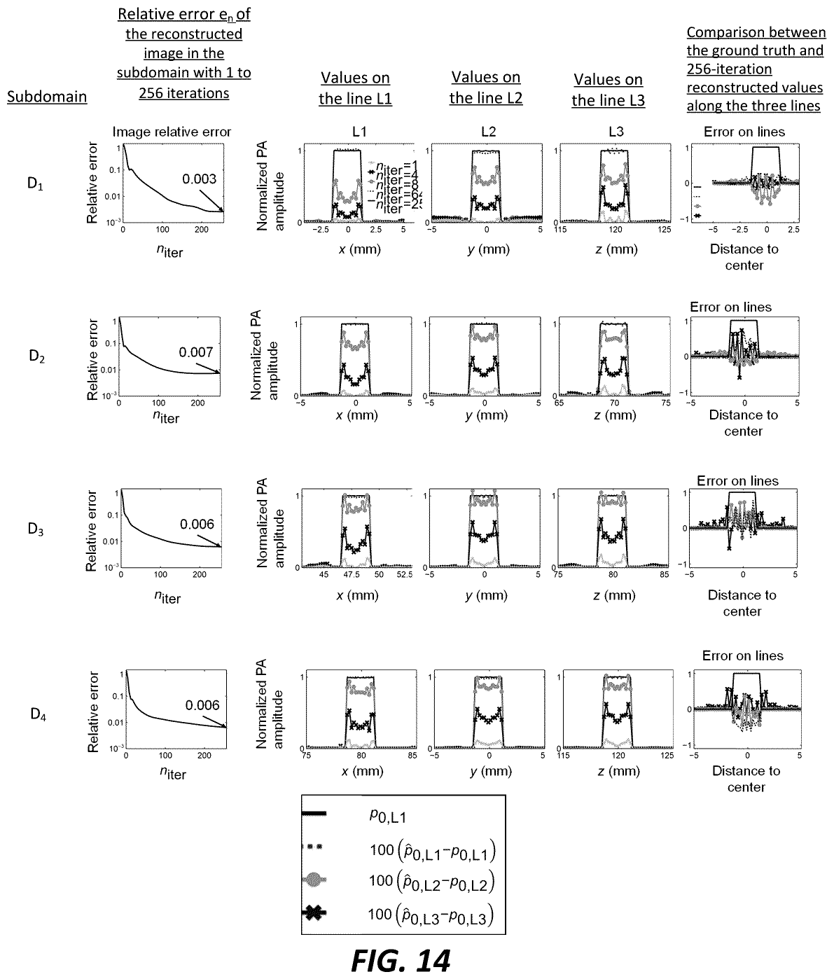

Based on the fast forward operator, one or more tomographic images can be reconstructed from the detected signals using an iterative reconstruction procedure. Images reconstructed iteratively using the fast forward operator from signals simulated by a traditional method (a slow operator) were observed to have negligible image-domain relative errors (maximum value of 0.007) as shown in FIG. 14 and discussed in Section III(E). FIG. 2B depicts an illustration of system matrix slicing in voxel indices (rectangular cuboids with dotted edges) and virtual element indices (thick arcs with boundaries).

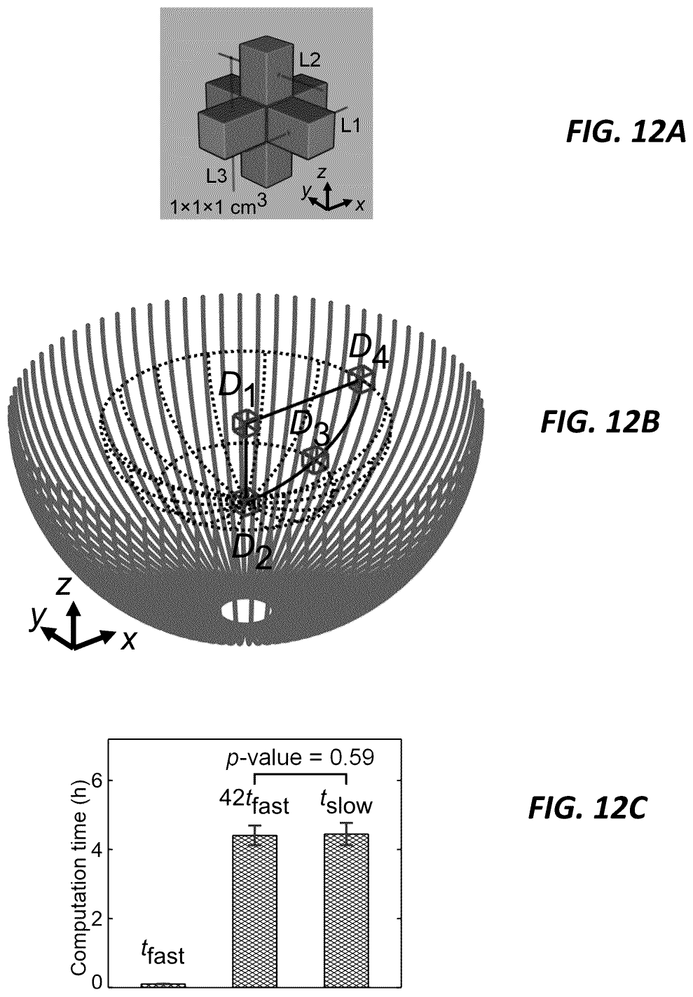

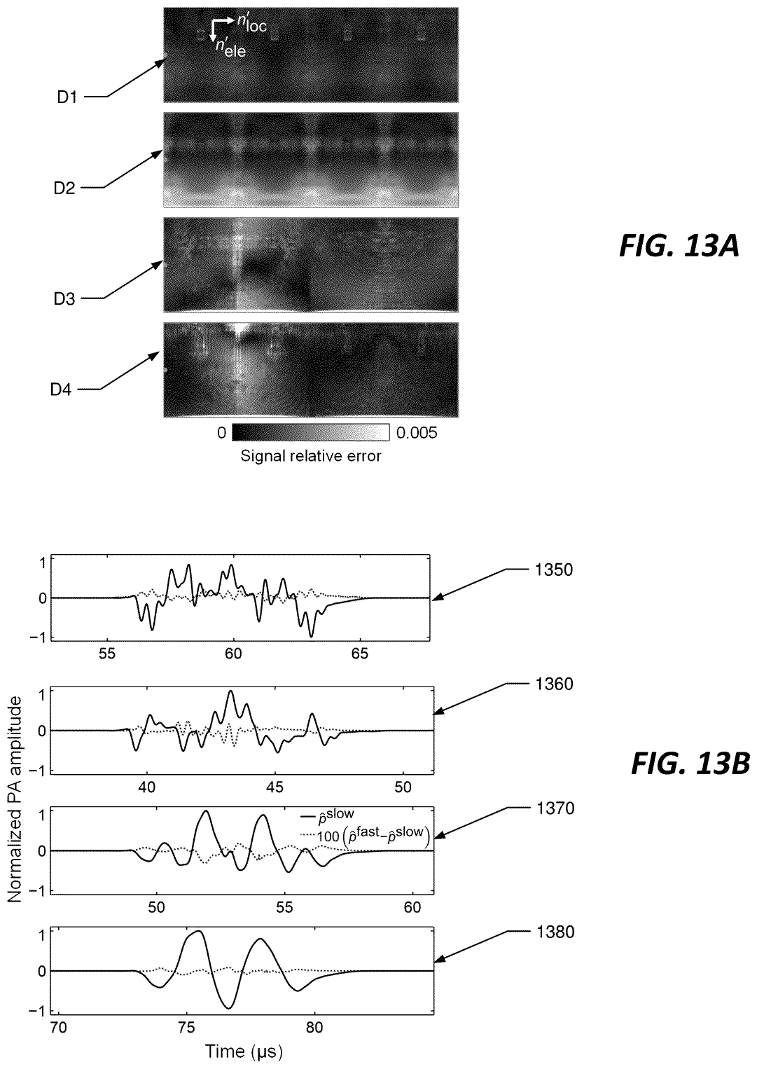

The original and compressed system matrices from a TISMC method may be described using a slow operator and a fast operator, respectively. The discretized operators are summarized in Eqns. 1 and 2. Through a forward simulation procedure, the compressed system matrix for the PACT system discussed above was shown to be approximately forty two (42) times as fast as the original system matrix with negligible signal-domain relative errors (maximum value of 0.005) as discussed in Section III (D) and shown in FIGS. 12A-C and FIGS. 13A-B. Further, for comparison, images were reconstructed using a nonnegativity-constrained iterative method described by Eqn. 33 with the compressed system matrix from signals simulated using the original system matrix. The images were observed to have negligible image-domain relative errors (maximum value of 0.007) as shown in FIG. 14 and discussed in Section III(E).

The implementation of the system matrix based on Eqn. 2 is not only efficient but also explicit, meaning subsets of the system matrix are directly accessible. For example, to obtain the signals from M′ voxels (enclosed in two rectangular cuboids with dotted edges in FIG. 2B) detected by N′ virtual elements (slender arcs between thick arcs in FIG. 2B), the relevant system matrix elements can be accessed directly and the computation in M′N′/MN of the time can be finished for M voxels and N elements.

According to various embodiments, a TISMC method performs piece-wise compression for each virtual sensor element of a tomographic system matrix. The piece-wise compression may only be performed once for a particular tomographic imaging system configuration as, for example, part of a calibration procedure. The piece-wise compression temporally shifts one or more of the responses to align arrival times and then group aligned responses for each sensor element. The TISMC method then performs an SVD procedure on the aligned responses to approximate the responses as linear combinations of a plurality of basis functions to obtain a compressed system matrix. The TISMC method may then be used to implement a fast forward operator to generate simulated virtual sensor signals. The fast forward operator method may then be used in an iterative reconstruction technique to reconstruct a tomographic image from detected signals.

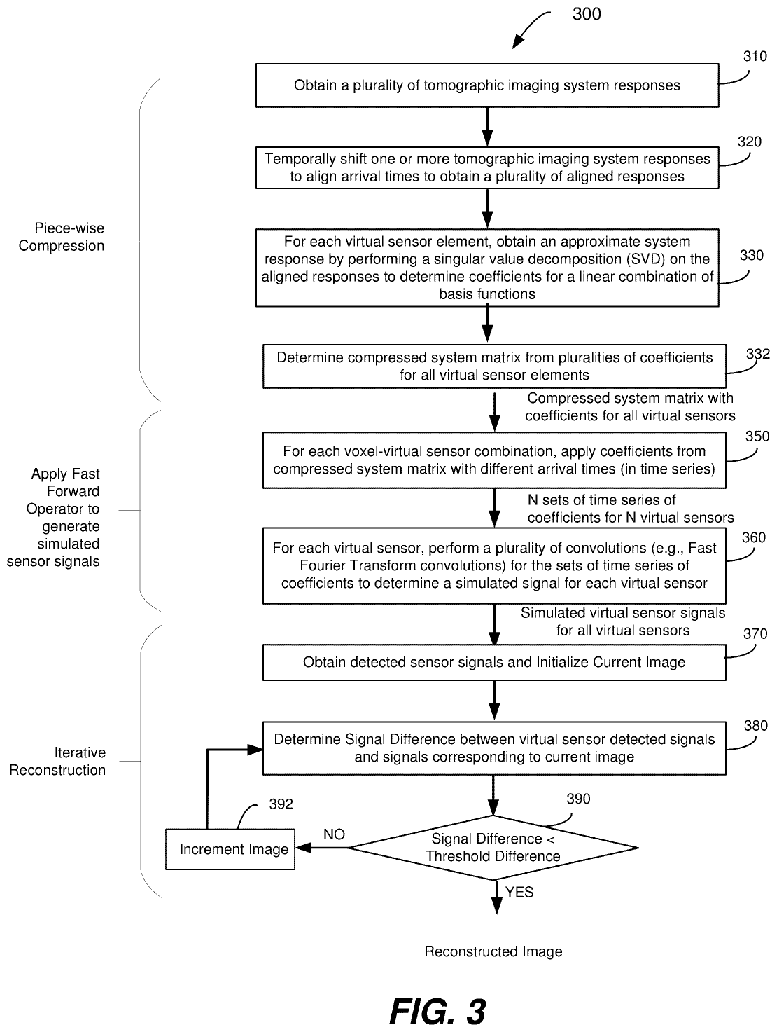

FIG. 3 depicts a flowchart illustrating operations of a TISMC method, according to various embodiments. One or more of the operations of the TISMC method may be performed by a computing device (e.g., computing device 2580 in FIG. 25A or computing device 2581 in FIG. 25B). The TISMC method includes a piece-wise compression process including operations 310, 320, and 330, a process that applies a fast forward operator to generate simulated sensor signals including operations 332 and 350, and a gradient descent iterative reconstruction process including operations 370, 380, 390, and 392. In another implementation, the TISMC method may employ an alternative iterative reconstruction process such as a FISTA procedure.

During an exemplary data acquisition phase of operation of a tomographic imaging system, sensor elements (e.g., transducers of one or more transducer arrays) are scanned to various detection locations at which signals are acquired from an object being imaged. As used herein, “virtual sensor elements” refer to the detection locations at which the sensor elements are located during signal acquisition. The detected signals may be stored to a buffer and/or recorded by one or more data acquisition systems of the tomographic imaging system. At operation 310, the TISMC method obtains a plurality of tomographic imaging system responses. The responses may be sensor signals detected during a calibration procedure when a calibration object is positioned for imaging in the tomographic imaging system. Alternatively, the responses may be simulated using the slow operator. The responses may be retrieved from memory or a buffer, or the signals may be received directly from the sensors of the tomographic imaging device.

At operation 320, the TISMC method temporally shifts one or more responses to align the arrival times of all responses. The arrival times of responses are determined by the voxel-sensor distances (i.e. distances between the voxel and the virtual sensor). As an example, in the illustrated example shown in FIG. 1B, the first responses 131, second response 132, . . . , MN response 133, are aligned to the same arrival time at dashed line 118.

At operation 330, the TISMC method performs a SVD procedure on the aligned responses to determine a plurality of coefficients for the linear combination of the basis functions that approximates the responses for each virtual sensor element. The plurality of basis functions may be obtained from the SVD procedure on the aligned responses. For example, the basis functions may be temporal singular vectors obtained from SVD procedure. An example of a plurality of basis functions are the first three temporal singular functions (e.g., three temporal singular functions 141, 142, and 143 in FIG. 1B). This SVD procedure is repeated for every virtual sensor element until a plurality of coefficients is determined for all the virtual sensor elements. At operation 332, the compressed system matrix is generated from the pluralities of coefficients 150 for all the virtual sensor elements.

At operation 350, for each voxel-virtual sensor combination, the TISMC method applies the coefficients from the compressed system matrix with the original arrival times to determine a time series of coefficients. For example, in the illustrated example shown in FIG. 2A, three time series of coefficients 201, 202, and 203 are obtained corresponding to the three temporal singular functions 141, 142, and 143 respectively. This operation is repeated for every voxel-virtual sensor combination until a time series of coefficients for all the voxel-virtual sensor combinations is determined.

At operation 360, for each virtual sensor, the TISMC method applies a plurality of convolutions (e.g., 3 convolutions for the three basis functions) to the sets of time series of coefficients to approximate a simulated signal for each virtual sensor. An example of a suitable convolution procedure that can be employed is convolution implemented by fast Fourier transform (FFT). This operation is repeated until a simulated signal is determined for a set of virtual sensors. In one embodiment, the operation is repeated until a simulated signal is determined for all virtual sensors. In another embodiment, the operation is repeated until a simulated signal is determined for every virtual sensor of a subset of all the virtual sensors.

At operation 370, the TISMC method obtains detected virtual sensor signals and initializes a current image. The detected sensor signals may be retrieved from memory or a buffer, or the signals may be received directly from the sensors of the tomographic imaging device. In one example, the voxels of the current image may be initialized with constant values.

At operation 380, the method determines a difference between the virtual sensor detected signals and the signals of the current image. At operation 390, if the difference between the virtual sensor detected signals and the signals of the current image is greater than, or equal to, a threshold difference, the image is incremented and the method returns to operation 380. An example of a threshold difference is 1% of the detected signals. If the difference between the virtual sensor detected signals and the signals of the current image is less than the threshold difference, a reconstructed image is output and the method ends.

### —Original Tomographic System Matrix and System Matrix Compressed by TISMC Method

Original and compressed tomographic imaging system matrices (compressed by a TISMC method according to an embodiment) can be described by a slow operator and a fast operator, respectively. The determination of the discretized operators is discussed in more detail in Section III(E) and is summarized below:

$${\hat{p}}_{n , l}^{slow} = {\sum }_{m = 1}^{M} {v}_{m} {h}_{n , m , l} {p}_{0 , m} , \qquad ( Eqn . 1 )$$

$$n = 1 , 2 , … , N , l = 1 , 2 , ⋯ , L ,$$

$$and$$

$${\hat{p}}_{n , l}^{fast} = {\sum }_{k = 1}^{K} {η}_{k , l ⋆ l} {\sum }_{m = 1}^{M} {v}_{m} {\hat{h}}_{lcl , k , n , m} {p}_{0 , m} \frac{1}{τ} [ \begin{matrix}( {t}_{{l}_{n , n} + 1} - \frac{‖ {r}_{m}^{′} - {r}_{n} ‖}{c} ) {δ}_{l , {l}_{n , m}} + \\ ( \frac{‖ {r}_{m}^{′} - {r}_{n} ‖}{c} - {t}_{{l}_{n , m}} ) {δ}_{l , {l}_{n , m} + 1}\end{matrix} ] , \qquad ( Eqn . 2 )$$

$$n = 1 , 2 , … , N , l = 1 , 2 , … , L .$$

The time variable is discretized as t₁=t₁+(l−1)τ, l=1, 2, . . . , L, where t₁ is the initial time, τ is the sampling step size, and L is the number of time points. In the operators, \hat{p}_{n,l} means the signal detected by the n-th virtual element at the time tₗ and is approximated by \hat{p}_{n,l}^{slow} and \hat{p}_{n,l}^{fast}, respectively; vₘ and p_{0,m} represents the volume and initial pressure of the m-th voxel, respectively; h_{n,m,l} is the n-th virtual element's response to the m-th voxel at tₗ; η_{k,l} is the k-th temporal singular function (obtained in SVD) evaluated at tₗ; K is the number of used singular value components; *ₗ means the discrete convolution for the time index l (implemented in FFT); ĥ_{lcl,k,n,m} is the k-th spatial singular function's value depending on the m-th voxel's location relative to the n-th virtual element; r′ₘ and rₙ are the coordinates of the m-th voxel and n-th virtual element, respectively; l_{n,m} denotes the temporal index such that

$${t}_{{l}_{n , m}} \leq \frac{‖ {r}_{m}^{′} - {r}_{n} ‖}{c} < {t}_{{l}_{n , m} + 1} ,$$

and the following Kronecker delta function is used

$${δ}_{l , {l}^{′}} = \{ \begin{matrix}0 , & l \neq {l}^{′} , \\ 1 , & l = {l}^{′} .\end{matrix} .$$

Although the numerical length of h_{n,m,l} (l=1, 2, . . . , L) is L, it has a smaller effective length, denoted as L′, for nonzero values. The symbols are summarized in Table 1 below. Numerical complexities of the slow and fast operators are O(NML′) and O(NMK)+O(N(L log₂ L)K), respectively. Here, the big O notations mean that there exist constants β₁, β₂, and β₃ (independent from NML′, NMK, and N(L log₂ L)K, respectively) such that the computations based on the slow and fast operators, respectively, can be done in times less than β₁NML′ and β₂NMK+β₃N(L log₂ L)K. The slow operator consists of only spatial integration; whereas, the fast operator consists of two layers of operations, and the terms β₂NMK and β₃N(L log₂ L)K are responsible for the inner layer spatial integration with ĥ_{lcl,k,n,m} and the outer layer temporal convolution with η_{k,l}, respectively. In numerical simulations with M=50×50×50, N=396×128, L=4096, L′=151, and K=3 discussed in Section III(D), it was observed that the fast operator based on Eqn. 2 has approximately forty two (42) times the speed of the slow operator based on Eqn. 1.

TABLE 1
Symbols for the forward operator and image reconstruction.

| | Definition as applied to | |
|---|---|---|
| Symbol | PACT system | Example Values for PACT system |
| M (m) | The number of voxels in the | In functional imaging, M = 180 × 240 × |
| | image (index) | 160 for numerical simulations and M = |
| | | 250 × 250 × 200 for in vivo experiments; |
| | | in motion correction, M = 224 × 224 × |
| | | 72 for numerical simulations and M = |
| | | 300 × 300 × 150 for in vivo experiments |
| v_{m,1}, v_{m,2}, and | Voxel sizes along the x-axis, | (v_{m,1}, v_{m,2}, v_{m,3}) = (0.1, 0.1, 0.1) (mm) |
| v_{m,3} (m) | y-axis, and z-axis, | for functional imaging |
| | respectively | (v_{m,1}, v_{m,2}, v_{m,3}) = (0.2, 0.2, 0.2) (mm) |
| | | for motion correction |
| N_{loc} (n_{loc}) and | Rotating location numbers of | In functional imaging, 4N_{loc} = 396 for |
| 4N_{loc} (n′_{loc}) | the four-arc array (index) and | dense sampling and 4N_{loc} = |
| | single-arc array (index), | 76, 40, 28, 20, 16, 12 for sparse sampling; |
| | respectively | in motion correction, 4N_{loc} = 288 for |
| | | numerical simulations and 4N_{loc} = 396 |
| | | for in vivo experiments |
| $\begin{matrix}{N}_{ele} ( {n}_{ele} ) and \\ \frac{{N}_{ele}}{4} ( {n}_{ele}^{′} )\end{matrix}$ | The numbers of transducer elements in the four-arc array (index) and single-arc array (index), respectively | $\begin{matrix}For numerical simulations , \frac{{N}_{ele}}{4} = 128 in \\ functional imaging and \frac{{N}_{ele}}{4} = 72 in \\ motion correction ; for in vivo experiments , \\ \frac{{N}_{ele}}{4} = 256\end{matrix}$ |
| a and b (m) | The length and width of each transducer element | a = 0.7 mm and b = 0.6 mm |
| N (n) | The number of virtual | N = N_{loc}Nₑₗₑ |
| | elements in the 2D array (index) | |
| τ (s) | The temporal sampling step | τ = 25 ns, which corresponds to a 40- |
| | size of each element | MHz sampling frequency |
| L | The temporal sampling | L = 4096 |
| | length of each element | |
| L′ | The effective length of the | L′ = 151 |
| | point source responses for | |
| | nonzero values | |
| Aₙ (m²) | The detection area of the n-th virtual element | Aₙ = ab = 0.42 mm², n = 1, 2, . . . , N |
| c (m · s⁻¹) | Speed of sound | C = 1.5 mm · us⁻¹ in numerical |
| | | simulations, tuned for in vivo experiments |
| K | The number of used singular | K = 3 |
| | components (Eqns. S16 and S27) | |

| | |
|---|---|
| vₘ (m³) | The volume of the m-th voxel: vm = v_{m,1}v_{m,2}v_{m,3} |
| r (m) | A location for ultrasonic signal detection |
| rₙ (m) | The center of the detection surface of the n-th virtual element |
| r′ (m) | A location in the image domain |
| r′ₘ (m) | The center of the m-th voxel in the image |
| t (s) | Time |
| tₗ (s) | The l-th sampling time |
| p₀(r′) (Pa) | The initial pressure at r′ |
| p(r, t) (Pa) | The pressure at location r and time t |
| h_{s,n}(r′, t) | The SIR of the n-th virtual element (Eqn. S3) |
| ĥ_{s,n}(r′, t) | The temporally shifted version of h_{s,n}(r′, t) (Eqn. S8) |
| h_{e,n} (t) (s⁻¹) | The EIR of the n-th virtual element (Eqn. S5) |
| hₑ(t) (s⁻¹) | The EIR shared by all virtual elements |
| \hat{p}(rₙ, t) (Pa) | The pressure detected by the n-th virtual element at time t (Eqn. S6) |
| hₙ(r′, t) (m⁻³) | The point source response (from r′) received by the n-th virtual element (Eqn. S7) |
| ĥₙ(r′, t) (m⁻³) | The temporally shifted version of hₙ(r′, t) (Eqn. S9) |
| Aₙ(m) | An orthonormal matrix formed by three vectors representing the local |
| | coordinates of the n-th virtual element (Eqn. S10) |
| r′_{lcl} (m) | A location in the local coordinate system of a virtual element |
| r′_{lcl,m}_{_{lcl}} (m) | The m_{lcl}-th location of interest in a virtual element's local coordinate system |
| ĥ_{s,lcl} (r′_{lcl}, t) | The temporally shifted SIR of a virtual element expressed in its local |
| | coordinates (Eqn. S12) |
| ĥ_{lcl}(r′_{lcl}, t) (m⁻³) | The temporally shifted point source response of a virtual element |
| | expressed in its local coordinates (Eqn. S13) |
| ĥ_{lcl,k} (r′_{lcl}) (m⁻³), | The k-th spatial singular function and the k-th temporal singular function, |
| and ηₖ (t) | respectively, in the SVD of ĥ_{lcl}(r′_{lcl}, t) (Eqn. S16) |
| ĥ_{lcl,m}_{_{lcl}}_{,l} (m⁻³) | The value of ĥ_{lcl}(r′_{lcl},ₘ_{_{lcl}}, tₗ) obtained through Eqn. S25 |
| h̅_{lcl,k,m}_{_{lcl}} (m⁻³) | The value of ĥ_{lcl,k}(r'_{lcl,m}_{_{lcl}}) obtained through Eqn. S27 |
| ĥ_{lcl,k,n,m} (m⁻³) | The value of ĥ_{lcl,k}((r'ₘ − rₙ)Aₙ) obtained through interpolation of h̅_{lcl,k,m}_{_{lcl}} |
| η_{k,l} | The value of ηₖ(tₗ) obtained through Eqn. S27 |
| h_{n,m,l} (m⁻³) | The value of hₙ(r′ₘ, tₗ) |
| p_{0,m} (Pa) | The value of p₀(r′ₘ) |
| \hat{p}_{n,l} (Pa) | The value of \hat{p}(rₙ, tₗ) |
| \hat{p}_{n,l}^{slow} (Pa) | An approximation of \hat{p}_{n,l} using the slow operator defined in Eqn. S26 |
| \hat{p}_{n,l}^{fast} (Pa) | An approximation of \hat{p}_{n,l} using the fast operator defined in Eqn. S30 |
| p₀ (Pa) | A vector formed by p_{0,m}, m = 1, 2, . . . , M |
| H | A system matrix formed by the coefficients in Eqn. S26 or Eqn. S30 |
| \hat{p} (Pa) | A vector formed by \hat{p}_{n,l}, n = 1, 2, . . . , N, l = 1, 2, . . . , L |
| \hat{p}₀ (Pa) | A vector formed by the reconstructed voxel values |
| λ (m) | The regularization parameter in the regularized optimization (Eqn. S34) |
| γ_{c} (Pa⁻¹) | The system-specific measurement calibration factor (ignored in practice) |
| \|p₀\|_{TV} (Pa · m⁻¹) | The TV norm of p₀ defined in Eqn. S35 |

The unit of an array represents the unit of its elements.

### B. Examples of Intra-Image Motion Correction Methods

According to various embodiments, an intra-image nonrigid motion correction method reconstructs an initial image without motion correction, decomposes the image into sub-images (subdomains of the image domain), estimates the motion of each sub-image for each location of the one or more sensor arrays of the tomographic imaging system (e.g., for every rotating location of a four-arc array of a PACT system), incorporates the motion information into the system matrix for a reconstruction of a motion corrected image, and, as needed, iterates the correction-reconstruction process to finalize the motion corrected image.

According to certain implementations, for intra-image nonrigid motion correction, motion parameters are incorporated into the tomographic system matrix and the motions and motion-corrected image simultaneously obtained by solving a dual-objective optimization problem (e.g., Eqn. 8) that minimizes the difference between the simulated signals from the subdomain and the detected signals. To describe intra-image motion in a tomographic image acquired by a PACT system according to an implementation, the number of elements of the four-arc transducer array are denoted as: Nₑₗₑ and the number of locations of the sensor array in a 3D imaging process are denoted as: N_{loc}.

In various implementations, an intra-image motion correction method decomposes the image domain, D, into rectangular-cuboid subdomains, D₁, D₂, . . . , D_{M}_{_{sub}}, satisfying Eqn. S37.

Other shapes may be used according to other implementations. The dimensions of subdomains may vary based on implementation. For example, for breast imaging, dimensions in the range of 1*1*1 cm\hat{ }3 to 3*3*3 cm\hat{ }3 may be used. As another example, for heart or brain imaging, dimensions in the range of 2*2*2 mm\hat{ }3 to 20*20*20 cm\hat{ }3 may be used. In some cases, the image domain may be defined by the field-of-view of the one or more sensor arrays of the tomographic imaging system.

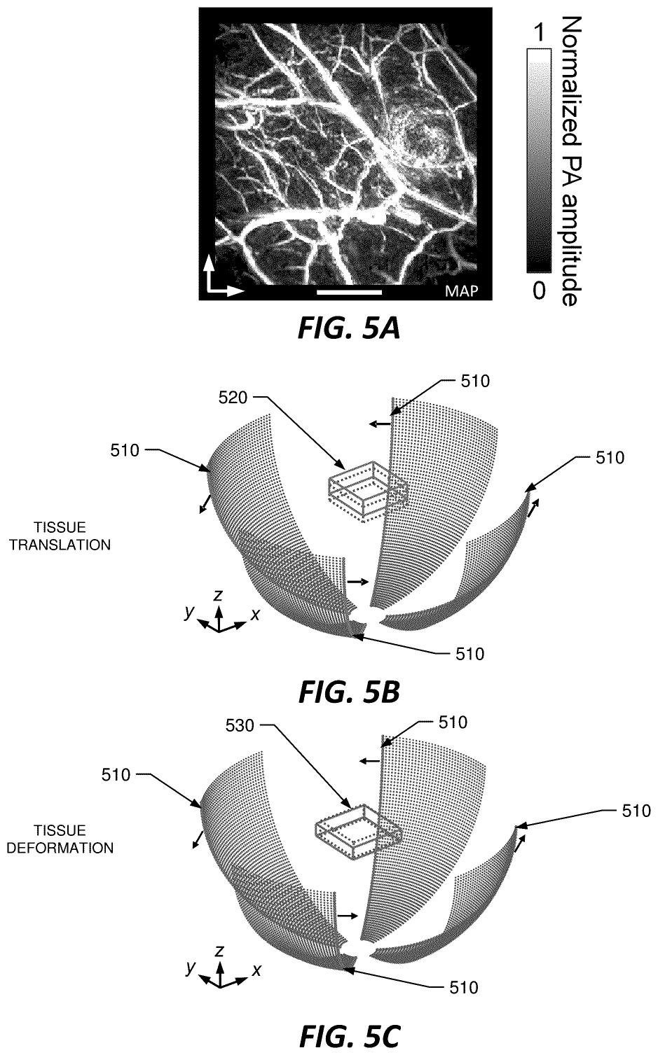

The deformations and rotations of each relatively small subdomain are generally small and each subdomain may be treated as rigid and only translates. The intra-image motion correction method discretizes the translations of each subdomain with translation step sizes, aₓ, a_{y}, and a_{z}, along an x-axis, y-axis, and z-axis (e.g., x-axis, y-axis, and z-axis shown in FIGS. 5B and 5C), respectively.

In one example, the translation steps of the subdomain of the m_{sub}-th subdomain, Dₘ_{_{sub}}, (with a subdomain center at r′_{c,m}_{_{sub}}) may be represented as translation step numbers of the m_{sub}-th subdomain along the x-axis, y-axis, and z-axis, kₓ(r′_{c,m}_{_{sub}}, n_{loc}), k_{y} (r′_{c,m}_{_{sub}}, n_{loc}), and k_{z}(r′_{c,m}_{_{sub}}, n_{loc}), respectively, when a sensor array moves to the N_{loc}-th location. Further, the following definitions are made: k(r′_{c,m}_{_{sub}}, n_{loc})=(kₓ(r′_{c,m}_{_{sub}}, n_{loc}), k_{y}(r′_{c,m}_{_{sub}}, n_{loc}), k_{z}(r′_{c,m}_{_{sub}}, n_{loc}))∈ [symbol] ³ and k={k(r′_{c,m}_{_{sub}}, n_{loc})∈ [symbol] ³|m_{sub}=1, 2, . . . , M_{sub}, n_{loc}=1, 2, . . . , N_{loc}}, which fully characterizes the intra-image motion in 3D tomographic imaging. Incorporating k into the system matrix using Eqns. S38 and S39, obtains the motion-incorporated system matrix, Hₖ. Then, motion correction may be expressed as the dual-objective optimization problem:

$$( {\hat{p}}_{0} , \hat{k} ) = {argmin}_{{p}_{0} ∈ {ℝ}^{M} , {p}_{0} \geq 0 , k ∈ {( {ℤ}^{3} )}^{M} {sub}^{N} loc} {γ}_{c} {‖ {H}_{k} {p}_{0} - \hat{p} ‖}^{2} + λ {\vert {p}_{0} \vert}_{T V} . \qquad ( Eqn . 8 )$$

This optimization problem may be decomposed into multiple subproblems (Eqns. S48, S49, and S50), which may be solved sequentially in multiple rounds of iterations (e.g., Gauss-Seidel-type iterations) according to various implementations. The iterations minimize the difference between the simulated signals from each subdomain and the detected signals.

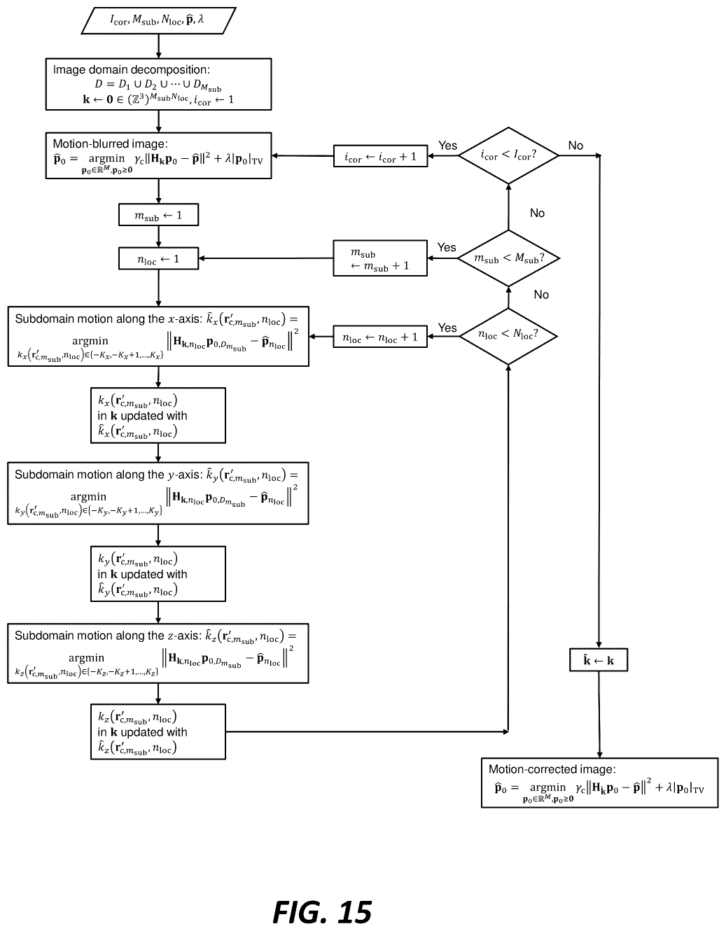

A detailed discussion of an example of an intra-image nonrigid motion correction method is shown in Section III(F). A workflow of the intra-image nonrigid motion correction method is shown in FIG. 15. Reference symbols are shown in Table 2.

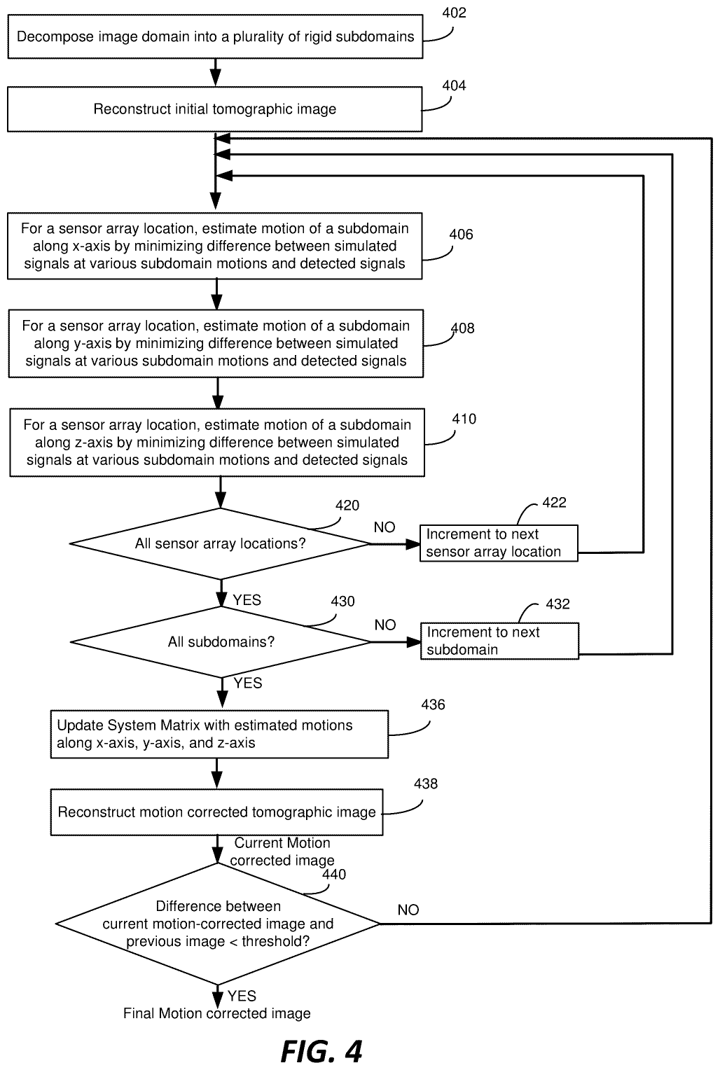

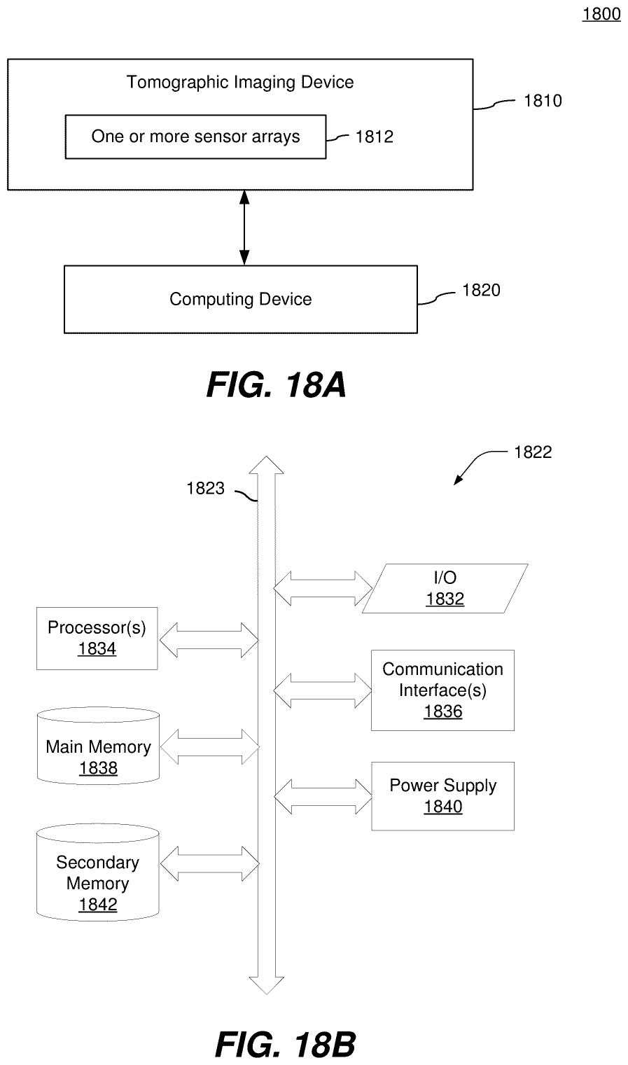

FIG. 4 depicts a flowchart illustrating operations of an intra-image nonrigid motion correction method, according to various embodiments. One or more of the operations of the intra-image nonrigid motion correction method may be performed by a computing device (e.g., computing device 1820 in FIG. 18A or computing device 1822 in FIG. 18B). The computing device may be in communication with a tomographic imaging system or another device to retrieve signals detected by one or more sensor arrays of the tomographic imaging system. In various examples, signals are from a single data acquisition run, as needed to generate a 3D tomographic image of a sample being imaged, are retrieved. During a single data acquisition run, the one or more sensor arrays are scanned to a plurality of sensor array locations. The signals from the one or more sensor arrays may be recorded and retrieved from memory or from a buffer, or the signals may be received directly from the sensor arrays of the tomographic imaging device. The field-of-view of the one or more sensors of the tomographic imaging system may define the total size of the imaging domain.

At operation 402, the intra-image nonrigid motion correction method decomposes the image domain of the signals detected by one or more sensor arrays of a tomographic imaging system into a plurality of subdomains. The number of subdomains and associated dimensions of the subdomains may vary based on implementation and the field-of-view of the tomographic imaging device. For example, for breast imaging, dimensions in the range of 1*1*1 cm\hat{ }3 to 3*3*3 cm\hat{ }3 may be used. As another example, for heart or brain imaging, dimensions in the range of 2*2*2 mm\hat{ }3 to 20*20*20 cm\hat{ }3 may be used.

At operation 404, the intra-image nonrigid motion correction method reconstructs a tomographic image of the sample. For example, at a first iteration, the intra-image nonrigid motion correction method may reconstruct a tomographic image (also referred to herein as a “motion-blurred image”) with the detected signals using an initial system matrix without motion correction. In any later iteration, the intra-image nonrigid motion correction method may reconstruct the tomographic image with the detected signals using a motion-corrected system matrix. An iterative reconstruction technique may be used such as FISTA or a gradient descent method.

At operation 406, the intra-image nonrigid motion correction method estimates a motion of a subdomain along an x-axis (e.g., x-axis shown in FIGS. 5B and 5C) for one sensor array location of the plurality of sensor array locations, e.g., all sensor array locations of the data acquisition run. In some implementations, a subset of all sensor array locations may be used. To estimate the subdomain motion along the x-axis, signals for various subdomain motions along the x-axis are simulated (e.g., using the numerical methods discussed in Section II(C)). The signals for the various subdomain motions are compared to the detected signals. The intra-image nonrigid motion correction method determines the simulated signals with the subdomain motion along the x-axis that most closely match the detected signals. The intra-image nonrigid motion correction method determines the estimated domain motion along the x-axis as the subdomain motion of the matching simulated signals.

At operation 408, the intra-image nonrigid motion correction method estimates a motion of a subdomain along a y-axis (e.g., y-axis shown in FIGS. 5B and 5C) for one sensor array location of the plurality of sensor array locations, e.g., all sensor array locations of the data acquisition run. In some implementations, a subset of all sensor array locations may be used. To estimate the subdomain motion along the y-axis, signals for various subdomain motions along the y-axis are simulated (e.g., using the numerical methods discussed in Section II(C)). The signals for the various subdomain motions are compared to the detected signals. The intra-image nonrigid motion correction method determines the simulated signals with the subdomain motion along the y-axis that most closely match the detected signals. The intra-image nonrigid motion correction method determines the estimated domain motion along the y-axis as the subdomain motion of the matching simulated signals.

At operation 410, the intra-image nonrigid motion correction method estimates a motion of a subdomain along a z-axis (e.g., z-axis shown in FIGS. 5B and 5C) for one sensor array location of the plurality of sensor array locations, e.g., all sensor array locations of the data acquisition run. In some implementations, a subset of all sensor array locations may be used. To estimate the subdomain motion along the z-axis, signals for various subdomain motions along the y-axis are simulated (e.g., using the numerical methods discussed in Section II(C)). The signals for the various subdomain motions are compared to the detected signals. The intra-image nonrigid motion correction method determines the simulated signals with the subdomain motion along the z-axis that most closely match the detected signals. The intra-image nonrigid motion correction method determines the estimated domain motion along the z-axis as the subdomain motion of the matching simulated signals.

Although the intra-image nonrigid motion correction method is described in operations 406, 408, and 410 as determining motions along an x-axis, a y-axis, and a z-axis, in other implementations, the intra-image nonrigid motion correction method may determine a motion along a single axis or along two axes. Moreover, in one implementation, a motion may be determined along a rotation axis.

At operation 420, if the motion (e.g., motion along x-axis, motion along y-axis, and motion along a z-axis) for the subdomain has not been estimated for all sensor array locations of the plurality of sensor array locations, the intra-image nonrigid motion correction method increments to the next sensor array location (operation 422) and repeats operations 406, 408 and 410 to estimate motion for the next sensor array location. If the motion for the subdomain has been estimated for all sensor array locations, the intra-image nonrigid motion correction method proceeds to operation 430.

At operation 430, if motion for all subdomains have not been estimated, the intra-image nonrigid motion correction method increments to the next subdomain (operation 432) and repeats operations 406, 408 and 410 to estimate motion for the next subdomain.

At operation 436, if the motions for all the subdomains have been estimated, the intra-image nonrigid motion correction method updates the tomographic system matrix with the estimated motions along the x-axis, along the y-axis, and along the z-axis. At operation 438, the intra-image nonrigid motion correction method reconstructs a motion-corrected image with the detected signals using the updated tomographic system matrix. An iterative reconstruction technique may be used such as FISTA or a gradient descent method.

At operation 440, the intra-image nonrigid motion correction method determines whether a difference between the current motion-corrected image and the previous reconstructed image (e.g., motion-corrected image of previous iteration or initial tomographic image reconstructed at operation 404) is less than a threshold. For example, 1% of the norm of the previous image. If the difference is more than, or equal to, the threshold, the intra-image nonrigid motion correction method returns to repeats operations 406, 408 and 410 based on the updated system matrix. If the difference is less than the threshold, the current motion corrected image is finalized as the final motion corrected image.

### C. Numerical Simulations of Intra-Image Motions

Numerical methods may be used to simulate translation- and deformation-induced motions in a tomographic image such as, for example, a tomographic image acquired by a PACT system. The results of a demonstration of an intra-image nonrigid motion correction method that uses numerical simulations to simulate images with translation- and deformation-induced motions is discussed in Section (D)(1).

To simulate translation- and deformation-induced motions, the values at

$$( x , y , z + {z}_{tra , {n}_{loc}} )$$

$$and$$

$$( \sqrt{{α}_{def , {n}_{loc}}} ( x - \frac{{x}_{0} + {x}_{1}}{2} ) , \sqrt{{α}_{def , {n}_{loc}}} ( y - \frac{{y}_{0} + {y}_{1}}{2} ) , \frac{1}{{α}_{def , {n}_{loc}}} ( z - {z}_{0} ) ) + ( \frac{{x}_{0} + {x}_{1}}{2} , \frac{{y}_{0} + {y}_{1}}{2} , {z}_{0} ) ,$$

respectively, in the n_{loc}-th numerical phantom were let to be the value at (x, y, z)∈[x₀, x₁]×[y₀, y₁]×[z₀, z₁] in a predefined numerical phantom, with the translation distances defined as:

$${z}_{tra , {n}_{loc}} = {A}_{tra} sin \frac{2 π ( {n}_{loc} - 1 ) {N}_{per}}{{N}_{loc}} , \qquad ( Eqn . 9 )$$

$${n}_{loc} = 1 , … , {N}_{loc}$$

and deformation ratios defined as:

$${α}_{def , {n}_{loc}} = 1 + {A}_{def sin} \frac{2 π ( {n}_{loc} - 1 ) {N}_{per}}{{N}_{loc}} , \qquad ( Eqn . 10 )$$

$${n}_{loc} = 1 , … , {N}_{loc} .$$

Here, Aₜᵣₐ and A_{def} are amplitudes of the translation distance and deformation ratio, respectively. In the forward simulations, signals from the n_{loc}-th numerical phantom are only detected by the transducer array at the n_{loc}-th location. We let x₁−x₀=44.8 mm, y₁−y₀=44.8 mm, z₁−z₀=14.4 mm, N_{loc}=72, Nₑₗₑ=4×72, and Nₚₑᵣ=0.5, 1, 2, 3 for all motion simulations, and use Aₜᵣₐ=0.2, 0.4, 0.6, 0.8, 1.0, 1.2 (mm) and A_{def}=0.02, 0.04, 0.06, 0.08, 0.10, 0.12, respectively, for translation and deformation simulations.

### D. Demonstration of Examples of Intra-Image Nonrigid Motion Correction Methods

Certain embodiments pertain to intra-image motion correction methods. FIG. 15 depicts a flowchart with workflow of operations of an intra-image motion correction method, according to an embodiment. FIG. 4 depicts a flowchart of operations of an intra-image motion correction method, according to various embodiments.

In this Section II(D), an example of an image motion correction method was used to correct for motion in numerical phantoms simulated with translation- and deformation-induced intra-image motions and to correct for heartbeat-induced motions in human breast imaging in vivo. The image motion correction method was demonstrated to successfully mitigate motion-induced artifacts and recover motion-compromised features in tomographic images.

1) Demonstrations with Numerical Phantom Simulations

An example of an intra-image motion correction method (e.g., intra-image motion correction method shown in FIG. 15 or intra-image motion correction method shown in FIG. 4) was demonstrated on numerical simulations of phantoms of intra-image motions generated by the technique described in Section II(C) above. From a numerical phantom obtained in 3D imaging of a human breast, numerical phantoms were simulated with translation- and deformation-induced intra-image motions, whose patterns are defined by Eqns. 9 and 10 respectively.

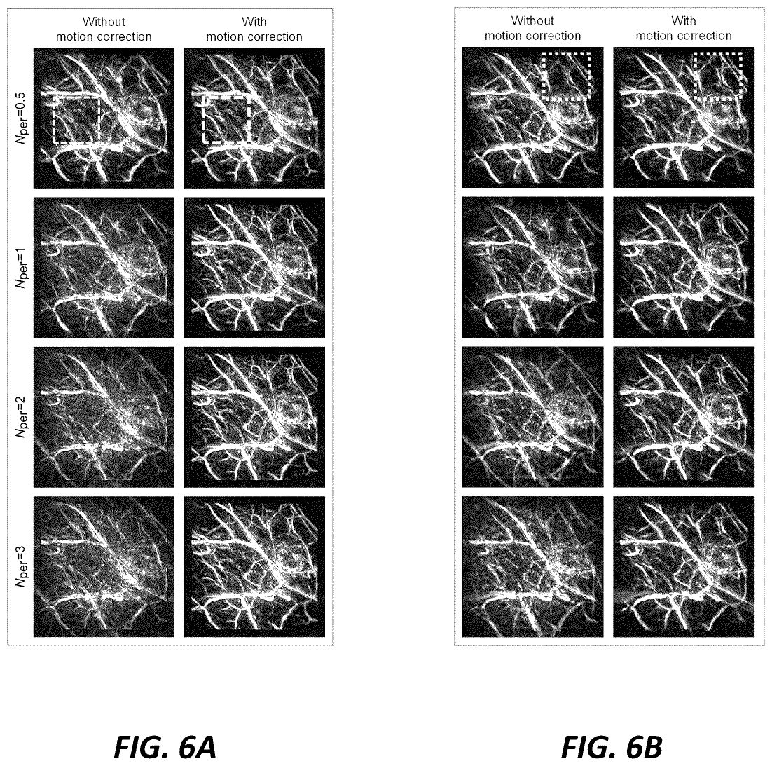

FIGS. 6A-B, and 7A-B show the results of intra-image nonrigid correction of the simulated motions induced by tissue translation and deformation. FIG. 5A depicts a MAP of a human breast image (e.g. human breast image subset with a volume of 44.8×44.8×14.4 mm³) along the z-axis, according to an embodiment. The scale bar is 1 cm.

FIGS. 5B-C depict rotation (denoted by arrows) of a four-arc transducer array with tissue translation and deformation. FIG. 5B depicts a schematic drawing of intra-image motions induced by tissue translation, according to an embodiment. FIG. 5C depicts a schematic drawing of intra-image motions induced by tissue deformation, according to an embodiment.

In FIG. 5B, an image 520 translates from a rectangular cuboid with dotted edges to one with solid edges as the four-arc transducer array rotates, with the passed virtual transducer element locations marked as dots, the latest transducer element locations 510 (denoted as thick lined curves), and the rotation direction indicated by arrows. In FIG. 5C, an image 530 deforms from a rectangular cuboid with dotted edges to one with solid edges as the four-arc transducer array rotates, with the passed virtual element locations marked as dots, the latest transducer element locations 510, and the rotation direction indicated by arrows.

During data acquisition phase of operation of a PACT system, a 900 rotation of the four-arc transducer array (e.g., N_{loc}=72, Nₑₗₑ=4×72) may allow the transducer elements to detect signals needed to form a 3D image, during which tissue motions (translation/deformation) may occur. The amplitudes of translation and deformation are controlled by the amplitude of the translation distance in translation-induced motions, Aₜᵣₐ, and the amplitude of the translation distance in deformation-induced motions, A_{def}, respectively, and the number of periods of the motion during a 900 rotation of the array is denoted as Nₚₑᵣ. The synchronized motions of the transducer array and numerical phantom of two examples were: Aₜᵣₐ=1.2 mm, Nₚₑᵣ=3 and A_{def}=0.12, Nₚₑᵣ=3 for translation and deformation, respectively, were determined. For detected transducer signals, the N_{loc} locations of the four-arc array (Nₑₗₑ elements) were reorganized to 4N_{loc} locations of the single arc array

$$( \frac{{N}_{e l e}}{4} elements )$$

with new indices n′_{loc}=1, 2, . . . , 4N_{loc} and n′ₑₗₑ=1, 2, . . . ,

$$\frac{{N}_{e l e}}{4} .$$

The signals for n′ₑₗₑ=18 and n′ₑₗₑ=72 (closer to the array-rotation axis) were determined. Additionally, the translation-induced motions with detected signals for Aₜᵣₐ=1.2 mm, Nₚₑᵣ=0.5, 1, 2, 3 were determined, and the deformation-induced motions with detected signals for A_{def}=0.12, Nₚₑᵣ=0.5, 1, 2, and 3 were determined.

The tomographic images were reconstructed from the simulated signals without and with motion correction and their MAPs were compared along the z-axis (translation-induced motions with Nₚₑᵣ=0.5, 1, 2, 3 and Aₜᵣₐ=0.2, 0.4, 0.6, 0.8, 1.0, 1.2 (mm)) and (deformation-induced motions with Nₚₑᵣ=0.5, 1, 2, 3 and A_{def}=0.02, 0.04, 0.06, 0.08, 0.10, and 0.12).

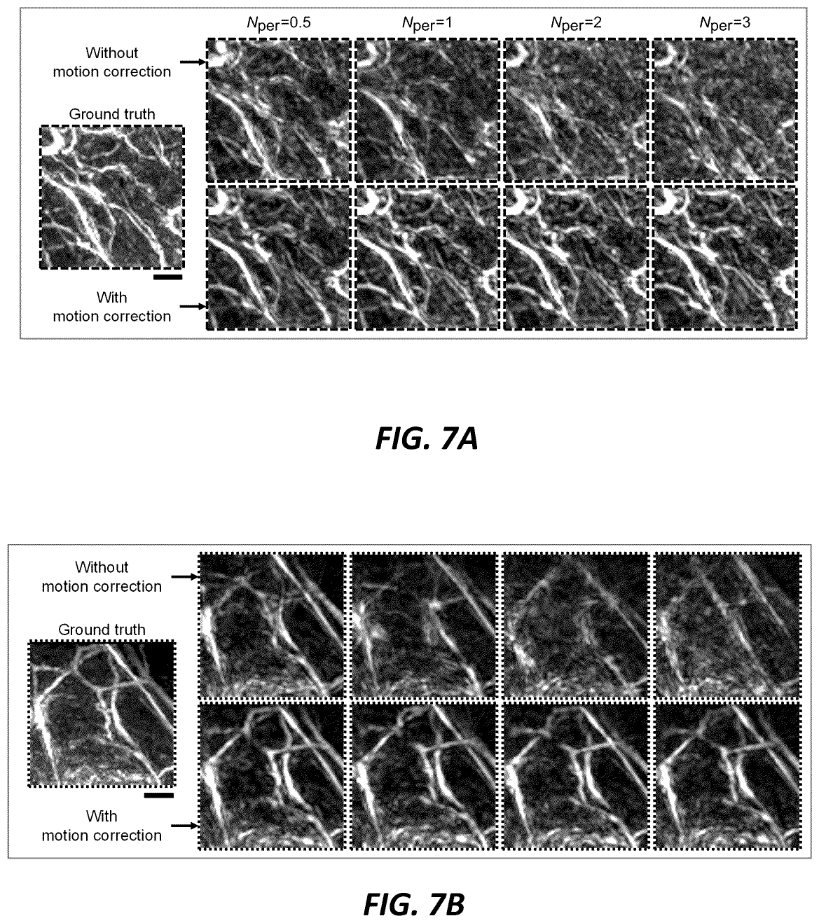

FIG. 6A depicts MAPs of the images reconstructed from the signals with motions induced by tissue translation (Aₜᵣₐ=0.6 mm) without and with motion correction for Nₚₑᵣ=0.5, 1, 2, and 3, according to an embodiment. FIG. 6B depicts MAPs of the images reconstructed from the signals with motions induced by deformation (A_{def}=0.06) without and with motion correction for Nₚₑᵣ=0.5, 1, 2, and 3, according to an embodiment. FIG. 7A depicts expanded views of subsets of the MAPs enclosed in dashed boxes in FIG. 6A and the ground truth for comparison, according to an embodiment. FIG. 7B depicts expanded views of subsets of the MAPs enclosed in dotted boxes in FIG. 6B and the ground truth for comparison, according to an embodiment.

The motion correction method used in this demonstration was shown to improve image quality for every set of signals as shown in the MAPS for Nₚₑᵣ=0.5, 1, 2, 3 in FIG. 6A (translation, Aₜᵣₐ=0.6 mm) and FIG. 6B (deformation, A_{def}=0.06). Moreover, the subsets of the MAPs are picked in dashed box and dotted box in FIGS. 6A and 6B, respectively, and the closeups of them shown in FIGS. 7A and 7B are compared with ground-truth MAPs. From the comparison, the motion correction method was shown to not only reduce the motion-induced artifacts but also reveal more true blood vessels, which match with those in the ground-truth images.

Although image-domain DNNs for motion correction may be powerful for mitigating artifacts, they are less capable of revealing features not observable in the original images. In practice, for a certain region in the tissue, if most of its motions have amplitudes greater than the image resolution, the detected signals from the region will have severe mismatches, which may cause feature loss in the reconstructed images. The fact that the motion correction method reveals these features proves its high tolerance to tissue motion and highlights the importance of system matrix manipulation in motion correction.

### 2) Demonstration of Correction for Heartbeat-Induced Motions in Human Breast Images

In another demonstration, an example of a motion correction method (e.g., intra-image motion correction method shown in FIG. 15 or the intra-image motion correction method shown in FIG. 4) was used to correct for heartbeat-induced motions in human breast images. In these experiments, signals were acquired using a PACT system (N_{loc}=99, Nₑₗₑ=4×256) from the left breast of a volunteer with breath holding (at 10 seconds) in four (4) in vivo experiments.

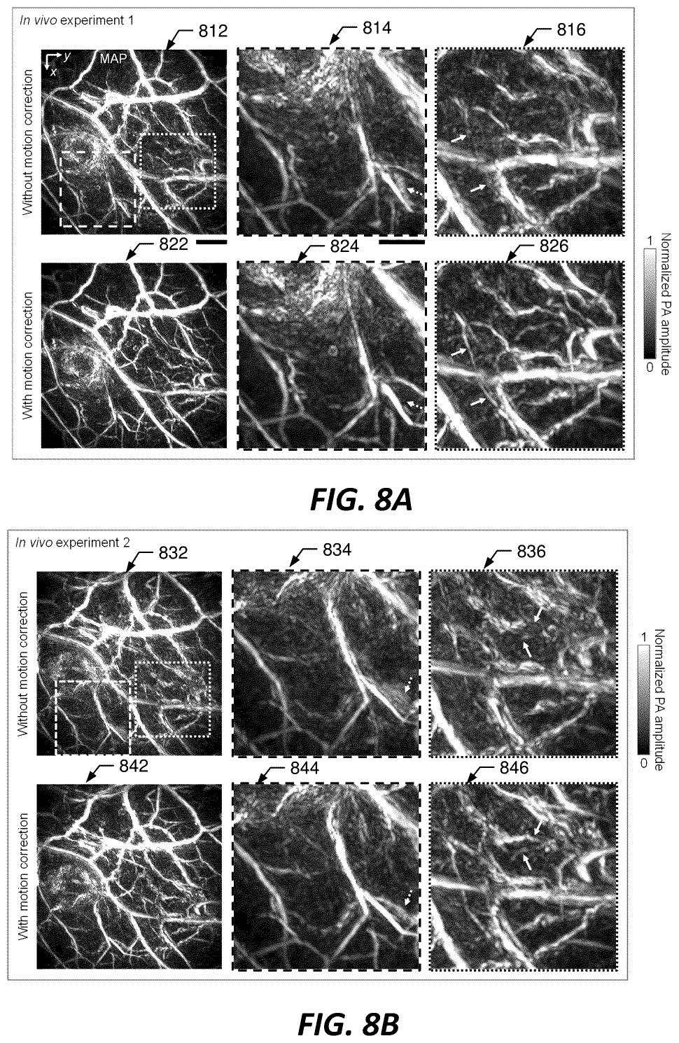

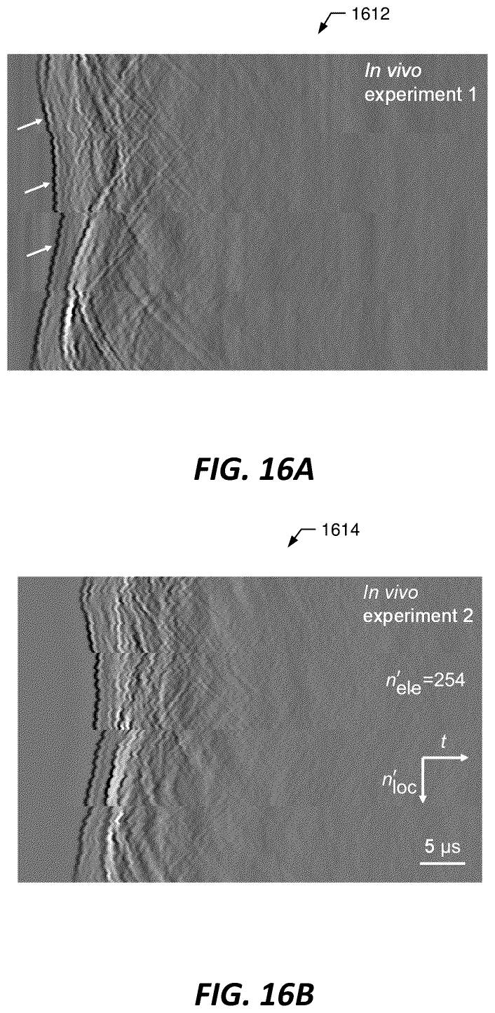

Samples of the signals taken in the first two experiments are shown in FIGS. 16A and 16B, respectively, from which the signals' temporal shifts caused by heartbeat-induced motions can be observed. Human breast images were reconstructed from signals taken in the first two experiments and the maximum amplitude projections (MAPs) of the images are shown in FIGS. 8A and 8B.

FIGS. 8A and 8B depict results of intra-image motion correction for human breast imaging for two in vivo imaging experiments of the same human breast. FIG. 8A depicts MAPs of a human breast image reconstructed without and with motion correction for the first experiment, according to an embodiment. FIG. 8B depicts MAPS of a human breast image reconstructed without and with motion correction for the second experiment, according to an embodiment. FIG. 8A includes a MAP 812 of a human breast image with a volume of 6×6×3 cm³ reconstructed without motion correction. FIG. 8A also includes a MAP 814 of a close up portion of the MAP 812 marked by a dashed box and a MAP 816 of a close up portion of the MAP 812 marked by a dotted box. FIG. 8A also includes MAPS 822, 824, and 826 of the images reconstructed with motion correction with the same signals as used to reconstruct the MAP image 812 and the close up portions in image 814 and 816. Examples of suppressed artifacts and enhanced features are highlighted in the images 814, 824 and 816, 826, respectively, by dotted and solid arrows. FIG. 8B includes a MAP 832 of a human breast image reconstructed without motion correction. FIG. 8A also includes a MAP 834 of a close up portion of the MAP 832 marked by a dashed box and a MAP 836 of a close up portion of the MAP 832 marked by a dotted box. FIG. 8B also includes MAPS 842, 844, and 846 of the images reconstructed with motion correction with the same signals as used to reconstruct the MAP image 832 and the close up portions in image 834 and 836. Examples of suppressed artifacts and enhanced features are highlighted in the images 834, 844 and 836, 846, respectively, by dotted and solid arrows.

For the first experiment, FIG. 8A depicts MAPs 812 and 822 of images reconstructed without and with motion correction, respectively. FIG. 8A also depicts a close-up subset of MAP 812 denoted by a dashed box in MAP 814 and another close-up subset of MAP 812 denoted by a dotted box in MAP 816. FIG. 8A also depicts a close-up subset of MAP 822 denoted by a dashed box in MAP 824 and another close-up subset of MAP 822 denoted by a dotted box in MAP 826. For the second experiment, FIG. 8B depicts MAPs 832 and 842 of images reconstructed without and with motion correction, respectively. FIG. 8B also depicts a close-up subset of MAP 832 denoted by a dashed box in MAP 834 and another close-up subset of MAP 832 denoted by a dotted box in MAP 836. FIG. 8B also depicts a close-up subset of MAP 842 denoted by a dashed box in MAP 844 and another close-up subset of MAP 842 denoted by a dotted box in MAP 846.

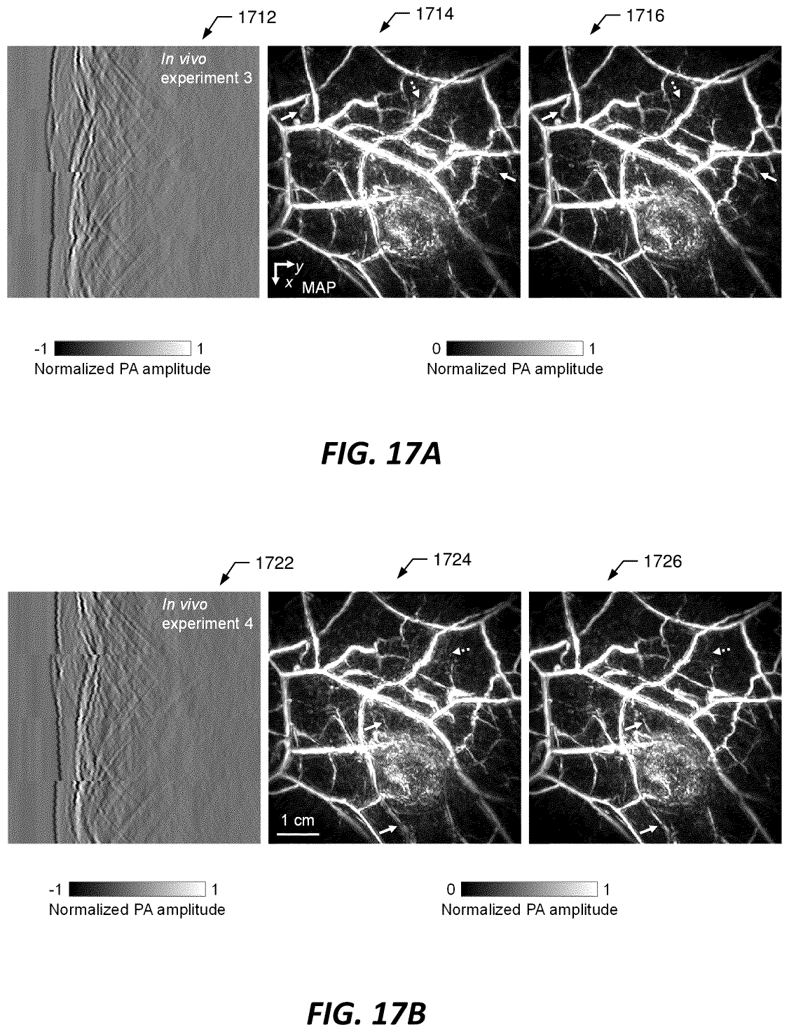

During the four experiments, four sets of signals (N_{loc}=99, Nₑₗₑ=4×256) from a human breast in vivo with heartbeat-induced motions and reconstructed images were acquired. FIG. 16A depicts an illustration of signals from the first human breast in vivo experiment. FIG. 16B depicts an illustration of signals from the second human breast in vivo experiment. The reconstructed images from the signals in FIGS. 16A and 16B are shown in FIGS. 8A and 8B, respectively. Temporal shifts in the signals shown in FIG. 16A are caused by quasiperiodic motion indicated by white arrows. FIG. 17A depicts an illustration 1712 of signals from the third human breast in vivo experiment, an image 1714 reconstructed without motion correction, and an image 1716 reconstructed with motion correction. FIG. 17B depicts an illustration 1722 of signals from the fourth human breast in vivo experiment, an image 1724 reconstructed without motion correction, and an image 1726 reconstructed with motion correction. Examples of the improvements due to motion correction are indicated by solid (feature enhanced) and dotted (artifacts suppressed) arrows.

It can be seen from the MAPs of the images from the first experiment that the motion correction method mitigates motion-induced artifacts (e.g., as shown in the region indicated by dotted arrows in MAPs 814, and 824 in FIG. 8A) and reveals motion-compromised features (e.g., as shown by the blood vessel indicated by solid arrows in the MAPS 816 and 826 in FIG. 8A). It can be seen from the MAPs of the images in the second experiment that motion correction recovers motion-compromised vessels, such as those indicated by solid arrows in MAPs 834, 844 and MAPs 836 and 846. Importantly, the compromised features the MAPs 814 and 816 are very different from those in MAPs 834 and 836 due to motions' different effects in the two experiments, although the breast deforms slightly between the two experiments. After motion correction, the features have high similarity as shown in MAPs 824 and 826 and MAPs 844 and 846, which validates that the features recovered by motion correction are accurate. Image qualities in the last two experiments are also improved by motion correction, as shown in images 1712, 1714, 1716 in FIG. 17A and images 1722, 1724, and 1726 in FIG. 17B, respectively.

### III. Supplemental Material

In Section III, examples of compression methods and intra-image motion correction methods are discussed. In some embodiments, a motion correction method includes a compression technique for compressing the system matrix. Implementing the compression technique, e.g., using Eqn. S30, may allow for efficient slicing of the system matrix with respect to image voxel indices and transducer element indices.

### A. Exemplary Fast Forward Operator for a TISMC Method Based on Singular Value Decomposition (SVD) and Fast Fourier Transform (FFT)

In this Section III(A), an example of a fast forward operator for a TISMC method, according to various embodiments, that includes singular value decomposition (SVD) and fast Fourier transform (FFT) is discussed for application with the PACT system described in Lin, L. et al. “High-speed three-dimensional photoacoustic computed tomography for preclinical research and clinical translation,” Nat. Commun. Vol. 12, pp. 1-10 (2021). Similar fast forward operators can be determined for application with other tomographic imaging systems, shown below in Section IV.

In a homogeneous medium, a photoacoustic wave can be expressed as:

$$p ( r , t ) = \frac{1}{4 π {c}^{2}} {\int }_{V} \frac{{p}_{0} ( {r}^{′} )}{‖ {r}^{′} - r ‖} \frac{\partial }{\partial t} δ ( t - \frac{‖ {r}^{′} - r ‖}{c} ) d {r}^{′} . \qquad ( Eqn . S 1 )$$

Here, p(r, t) is the pressure at location r and time t, c is the speed of sound (SOS), V is the volumetric space occupied by the tissue, and p₀(r′) is the initial pressure at r′. Assume the PACT system includes N finite-size ultrasonic transducer elements. For the n-th transducer element at rₙ with detection surface Sₙ, the average pressure on the detection surface at time t is expressed as

$$\tilde{p} ( {r}_{n} , t ) = \frac{1}{{A}_{n}} {\int }_{{S}_{n}} p ( r , t ) {da}_{n} ( r ) . \qquad ( Eqn . S 2 )$$

Here, Aₙ denotes the area of the surface Sₙ. The spatial impulse response (SIR) in Eqn. S2 is denoted as

$${h}_{s , n} ( {r}^{′} , t ) = {\int }_{{S}_{n}} \frac{δ ( t - \frac{‖ {r}^{′} - r ‖}{c} )}{2 π c ‖ {r}^{′} - r ‖} {da}_{n} ( r ) . \qquad ( Eqn . S 3 )$$

Then Eqn. S2 becomes:

$$\tilde{p} ( {r}_{n} , t ) = \frac{1}{2 c} {\int }_{V} {p}_{0} ( {r}^{′} ) \frac{1}{{A}_{n}} \frac{\partial }{\partial t} [ {\int }_{{S}_{n}} \frac{δ ( t - \frac{‖ {r}^{′} - r ‖}{c} )}{2 π c ‖ {r}^{′} - r ‖} {da}_{n} ( r ) ] {dr}^{′} = \frac{1}{2 c} {\int }_{V} {p}_{0} ( {r}^{′} ) \frac{1}{{A}_{n}} \frac{\partial }{\partial t} {h}_{s , n} ( {r}^{′} , t ) {dr}^{′} . \qquad ( Eqn . S 4 )$$

The electric impulse response (EIR) of the n-th transducer is denoted as h_{e,n}(t) and the transducer's response using temporal convolution *ₜ is expressed as:

$$\hat{p} ( {r}_{n} , t ) = \tilde{p} ( {r}_{n} , t ) {*}_{t} {h}_{e , n} ( t ) . \qquad ( Eqn . S 5 )$$

Substituting Eqn. S4 into Eqn. S5 yields:

$$\hat{p} ( {r}_{n} , t ) = \frac{1}{2 c} {\int }_{V} {p}_{0} ( {r}^{′} ) \frac{1}{{A}_{n}} \frac{\partial }{\partial t} {h}_{s , n} ( {r}^{′} , t ) {dr}^{′} {*}_{t} {h}_{e , n} ( t ) = {\int }_{V} {p}_{0} ( {r}^{′} ) \frac{{h}_{e , n}^{′} ( t ) {*}_{t} {h}_{s , n} ( {r}^{′} , t )}{2 {cA}_{n}} {dr}^{′} = {\int }_{V} {p}_{0} ( {r}^{′} ) {h}_{n} ( {r}^{′} , t ) {dr}^{′} . \qquad ( Eqn . S 6 )$$

Here, the prime in h′_{e,n}(t) denotes the time derivative, and hₙ denotes the point source response per unit initial pressure per unit infinitesimal tissue volume received by a finite-size transducer element:

$${h}_{n} ( {r}^{′} , t ) = \frac{{h}_{e , n}^{′} ( t ) {*}_{t} {h}_{s , n} ( {r}^{′} , t )}{2 {cA}_{n}} . \qquad ( Eqn . S 7 )$$

For convenience in the following discussion, t h_{s,n}(r′, t) and hₙ(r′, t) for r′ are temporally shifted such that time 0 aligns with the onset of the nonzero signal (arrival time) received by the center of the n-th transducer element rₙ:

$${\hat{h}}_{s , n} ( {r}^{′} , t ) = {h}_{s , n} ( {r}^{′} , t + \frac{‖ {r}^{′} - {r}_{n} ‖}{c} ) = {\int }_{{S}_{n}} \frac{δ ( t + \frac{‖ {r}^{′} - {r}_{n} ‖}{c} - \frac{‖ {r}^{′} - r ‖}{c} )}{2 π c ‖ {r}^{′} - r ‖} {da}_{n} ( r ) \qquad ( Eqn . S 8 )$$

$$and$$

$${\hat{h}}_{n} ( {r}^{′} , t ) = {h}_{n} ( {r}^{′} , t + \frac{‖ {r}^{′} - {r}_{n} ‖}{c} ) = \frac{{h}_{e , n}^{′} ( t ) {*}_{t} {h}_{s , n} ( {r}^{′} , t + \frac{‖ {r}^{′} - {r}_{n} ‖}{c} )}{2 {cA}_{n}} = \frac{{h}_{e , n}^{′} ( t ) {*}_{t} {\hat{h}}_{s , n} ( {r}^{′} , t )}{2 {cA}_{n}} . \qquad ( Eqn . S 9 )$$

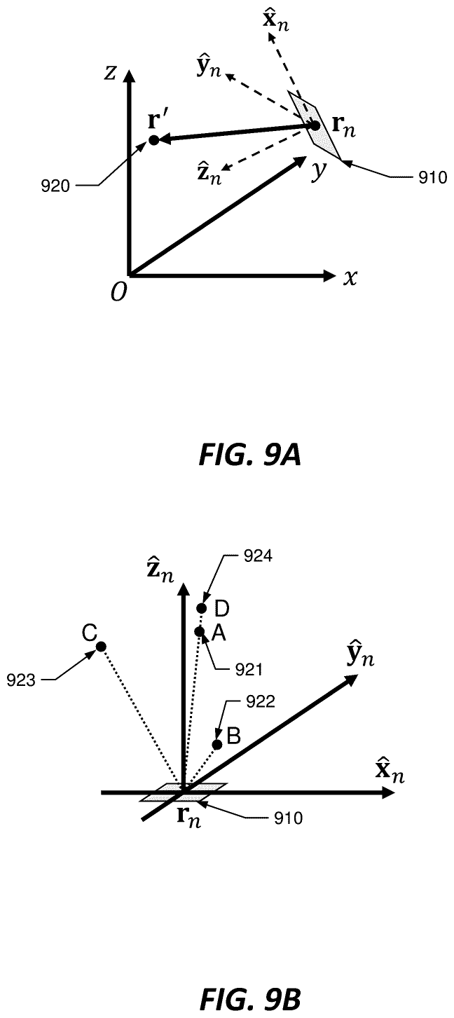

Next, the SIR and point source response are expressed in the local coordinates of the transducer elements. Each transducer element in this example has a flat rectangular detection surface with a length a of 0.7 mm and a width b of 0.6 mm, yielding Aₙ=ab=0.42 mm², n=1, 2, . . . , N. These transducer elements also have the same EIR: h_{e,n}(t)=hₑ(t), n=1, 2, . . . , N, and the measurement of hₑ(t) will be discussed in Section III(B). The local coordinates of the n-th transducer element are defined using the center the detection surface as the origin, the length direction as the x-axis, the width direction as the y-axis, and the normal direction (toward the detection region) as the z-axis. The x-axis and y-axis are selected to let the coordinates satisfy the right-hand rule. The three axes of the local coordinates are expressed as three vectors of unit length: \hat{x}ₙ, ŷₙ, and \hat{z}ₙ (shown in FIG. 9A), which form an orthonormal matrix:

$${A}_{n} = ( {\hat{x}}_{n}^{T} , {\hat{y}}_{n}^{T} , {\hat{z}}_{n}^{T} ) . \qquad ( Eqn . S 10 )$$

Locations r′, rₙ, and r in the global coordinates correspond to the locations r′_{lcl}, 0, and r_{lcl} in the local coordinates of the n-th transducer element. Coordinate transformations yield r′_{lcl}=(r′−rₙ)Aₙ and r_{lcl}=(r−rₙ)Aₙ. These global and local coordinates satisfy ∥r′−rₙ∥=∥r_{lcl}∥ and ∥r′−r∥=∥r′_{lcl}−r_{lcl}∥ due to the orthonormality of the transformations. The detection surface (Sₙ in the global coordinates) is denoted in the local coordinates as S_{n,lcl}, and the local Leibniz notation is denoted as da_{n,lcl}(r_{lcl})=daₙ(r). Thus, in the local coordinates of the n-th transducer element, Eqn. S8 is expressed as:

$${\hat{h}}_{s , n} ( {r}^{′} , t ) = {\int }_{{S}_{n , lcl}} \frac{δ ( t + \frac{‖ {r}_{lcl}^{′} ‖}{c} - \frac{‖ {r}_{lcl}^{′} - {r}_{lcl} ‖}{c} )}{2 π c ‖ {r}_{lcl}^{′} - {r}_{lcl} ‖} {da}_{n , lcl} ( {r}_{lcl} ) , \qquad ( Eqn . S 11 )$$

$${r}_{lcl}^{′} = ( {r}^{′} - {r}_{n} ) {A}_{{r}_{n}} , n = 1 , 2 , ... , N .$$

All transducer elements are geometrically identical and have the same local coordinates, meaning S_{n,lcl}=S_{n′,lcl} and da_{n,lcl}(r_{lcl})=da_{n′,lcl}(r_{lcl}) for n, n′∈{1, 2, . . . , N}. Define S_{lcl}=S_{1,lcl}, da_{lcl} (r_{lcl})=da_{1,lcl}(r_{lcl}), and rewrite Eqn. S11 as:

$${\hat{h}}_{s , lcl} ( {r}_{lcl}^{′} , t ) = {\int }_{{S}_{lcl}} \frac{δ ( t + \frac{‖ {r}_{lcl}^{′} ‖}{c} - \frac{‖ {r}_{lcl}^{′} - {r}_{lcl} ‖}{c} )}{2 π c ‖ {r}_{lcl}^{′} - {r}_{lcl} ‖} {da}_{lcl} ( {r}_{lcl} ) , \qquad ( Eqn . S 12 )$$

which is now independent of the transducer element index n. Replacing h′_{e,n}(t) and ĥ_{s,n}(r′, t) with h′ₑ(t) and ĥ_{s,lcl}(r′_{lcl}, t), respectively, in Eqn. S9, define:

$${\hat{h}}_{lcl} ( {r}_{lcl}^{′} , t ) = \frac{{h}_{e}^{′} ( t ) {*}_{t} {\hat{h}}_{s , lcl} ( {r}_{lcl}^{′} , t )}{2 cab} . \qquad ( Eqn . S 13 )$$

Thus, it is needed to calculate only the values of ĥ_{s,lcl}(r′_{lcl}, t) and ĥ_{lcl}(r′_{lcl}, t), then obtain the values of ĥ_{s,n}(r′, t) and ĥₙ(r′, t) through coordinate transformation:

$${\hat{h}}_{s , n} ( {r}^{′} , t ) = {\hat{h}}_{s , lcl} ( {r}_{lcl}^{′} , t ) \qquad ( Eqn . S 14 )$$

$$and$$

$${\hat{h}}_{n} ( {r}^{′} , t ) = \frac{{h}_{e , n}^{′} ( t ) {*}_{t} {\hat{h}}_{s , n} ( {r}^{′} , t )}{2 {cA}_{n}} = \frac{{h}_{e}^{′} ( t ) {*}_{t} {\hat{h}}_{s , lcl} ( {r}_{lcl}^{′} , t )}{2 cab} = {\hat{h}}_{lcl} ( {r}_{lcl}^{′} , t ) , \qquad ( Eqn . S15 )$$

respectively, with r′_{lcl}=(r′−rₙ)Aₙ for n=1, 2, . . . , N. Through these relations, both the SIR and the point source response are expressed in the local coordinates.

FIG. 9A depicts an illustration of an n-th transducer element (a black rectangular centered at rₙ) 910, a single point source 920, and n-th transducer element local coordinate system with axes \hat{x}n, ŷₙ, and \hat{z}ₙ, according to an embodiment. FIG. 9B depicts an illustration of four point sources: a first point source 921, a second point source 922, a third point source 923, and a fourth point source 924 in the local coordinate system of the n-th transducer element 910, according to an embodiment. Points A, D, and rₙ are on the same line.

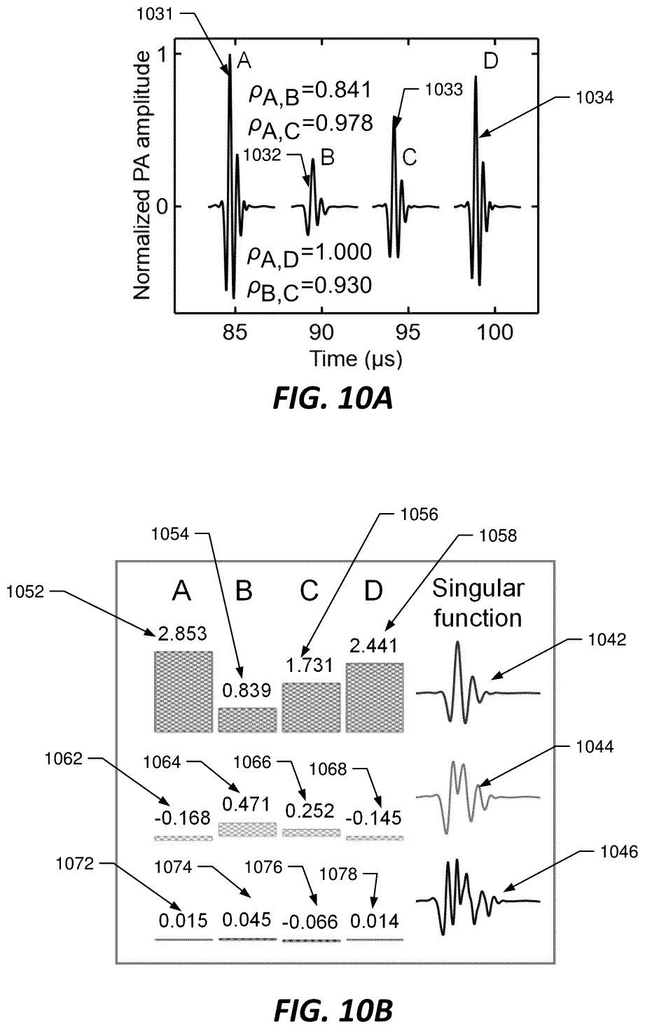

FIG. 10A depicts a graph of the responses 1031, 1032, 1033, and 1034 of the transducer element 910 in to the signals from the four point sources 921, 922, 923, and 924 in FIG. 9B and the PCC between every two responses. The time differences between responses are due to the differences in distances between voxel and transducer element. FIG. 10B depicts an illustration of four responses to the signals from the four point sources 921, 922, 923, and 924 in FIG. 9B using linear combinations (coefficients visualized with bars) of three temporal singular functions 1042, 1044, and 1046, respectively, based on a SVD procedure according to an embodiment. The illustration shows coefficients 1052, 1054, 1056, and 1058 for the first linear combination for the first singular function 1042, coefficients 1062, 1064, 1066, and 1068 for the second linear combination for the second singular function 1044, and coefficients 1072, 1074, 1076, and 1078 for the third linear combination for the third singular function 1046.

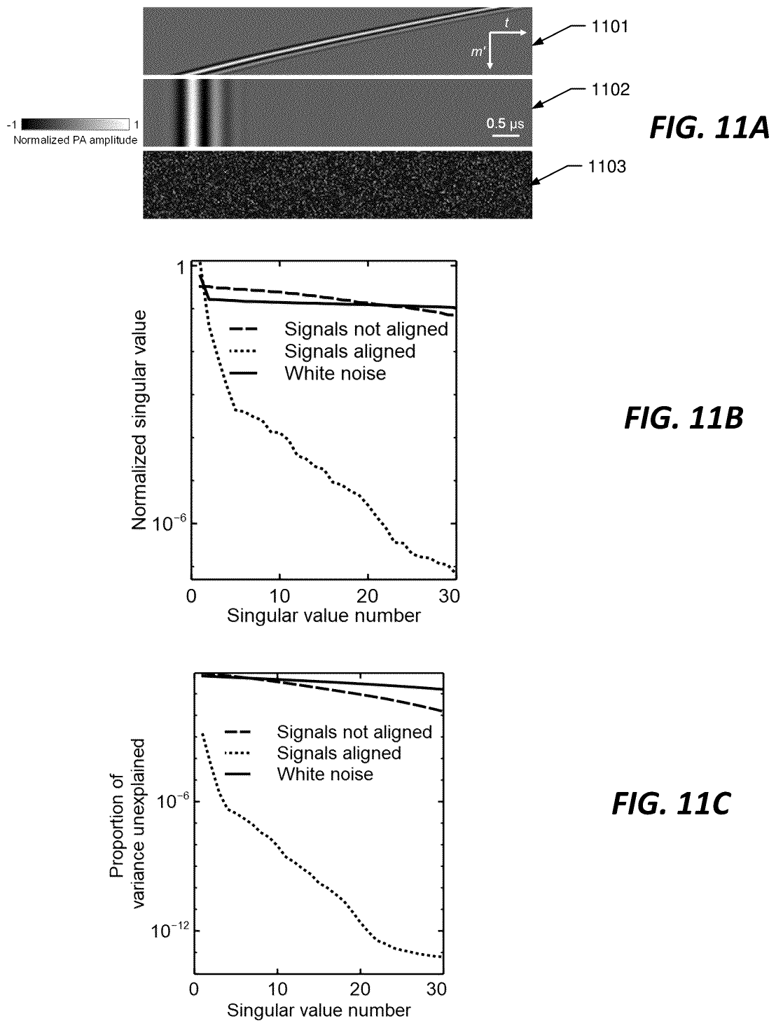

FIG. 11A depicts an illustration 1101 of response of a transducer element to fifty (50) point sources with decreasing distances to the element, an illustration 1102 of the temporally-shifted form which aligns the non-zero signals in time, and an illustration 1103 of white noise responses of the same size of as those in illustration 1102, according to an embodiment. FIG. 11B depicts a graph with normalized singular values in the SVDs of the signals in illustrations 1101, 1102, and 1103, according to an embodiment. FIG. 11C depicts a graph with proportions of the variances unexplained in the SVDs of the signals in illustrations 1101, 1102, and 1103, according to an embodiment.

In the illustrated example shown in FIG. 9B, the signals detected by a transducer element can be visualized by picking four point sources four point sources, labeled as A, B, C, and D, respectively, in the local coordinate system of the n-th transducer element, with points A, D, and the element center rₙ on the same line. FIG. 10A depicts a plot of the element's responses to the signals from the point sources 921, 922, 923, and 924 in FIG. 9B. ρ_{A,B} denotes the Pearson correlation coefficient (PCC) between the responses corresponding to A and B. A direct implementation of the forward operator based on Eqn. S6 is computationally intensive. Although ρ_{A,D}=1, due to the effects of SIR, ρ_{A,B}, ρ_{A,C}, and ρ_{B,C} are less than 1, indicating the signals from points A, B, and C are not shift-invariant; thus, an efficient temporal convolution with one kernel function cannot yield the detected signals accurately.

To accelerate the forward operator, the spatial and temporal dimensions of ĥ_{lcl}(r′_{lcl}, t) may be decoupled using singular value decomposition (SVD) while keeping only the dominant components:

$${\hat{h}}_{lcl} ( {r}_{lcl}^{′} , t ) \approx {\sum }_{k = 1}^{K} {\hat{h}}_{lcl , k} ( {r}_{lcl}^{′} ) {η}_{k} ( t ) . \qquad ( Eqn . S 16 )$$

Here, ĥ_{lcl,k}(r′_{lcl}) and ηₖ denote the k-th spatial singular function and the k-th temporal singular function, respectively; and the first K terms are used to approximate the whole series. Combining Eqns. S9, S15, and S16, the following can be obtained:

$${h}_{n} ( {r}^{′} , t ) = {\hat{h}}_{n} ( {r}^{′} , t - \frac{‖ {r}^{′} - {r}_{n} ‖}{c} ) = {\hat{h}}_{lcl} ( ( {r}^{′} - {r}_{n} ) {A}_{n} , t - \frac{‖ {r}^{′} - {r}_{n} ‖}{c} ) \approx {\sum }_{k = 1}^{K} {\hat{h}}_{lcl , k} ( ( {r}^{′} - {r}_{n} ) {A}_{n} ) {η}_{k} ( t - \frac{‖ {r}^{′} - {r}_{n} ‖}{c} ) . \qquad ( Eqn . S 17 )$$

Substituting Eqn. 17 into Eqn. S6, the following can be obtained:

$$\hat{p} ( {r}_{n} , t ) \approx {\int }_{V} {p}_{0} ( {r}^{′} ) {\sum }_{k = 1}^{K} {\hat{h}}_{lcl , k} ( ( {r}^{′} - {r}_{n} ) {A}_{n} ) {η}_{k} ( t - \frac{‖ {r}^{′} - {r}_{n} ‖}{c} ) {dr}^{′} = {\sum }_{k = 1}^{K} {η}_{k} ( t ) {*}_{t} {\int }_{V} {p}_{0} ( {r}^{′} ) {\hat{h}}_{lcl , k} ( ( {r}^{′} - {r}_{n} ) {A}_{n} ) δ ( t - \frac{‖ {r}^{′} - {r}_{n} ‖}{c} ) {dr}^{′} , \qquad ( Eqn . S 18 )$$

$$n = 1 , 2 , ... , N , t \geq 0.$$

As shown in this equation, the temporal variable is split from the spatial integrals, which allows for a fast implementation of the forward operator.

For an initial demonstration, Eqn. S16 with K=3 is applied to the responses 1031, 1032, 1033, and 1034 in FIG. 10A. FIG. 10B depicts an illustration of the three temporal singular functions η₁(t) 1042, η₁₂(t) 1044, η₃(t) 1046, respectively, and the values (coefficients) of the spatial singular functions 1042, 1044, 1046 are visualized as bars.

In various embodiments, a TISMC method includes temporal shifting for alignment. An example of temporal shifting for SVD is described in Eqns. S8 and S9. In one example, fifty (50) point sources were selected with decreasing distances to a transducer element and the element's independent responses to them were visualized as shown in illustration 1101 in FIG. 11A.

The temporally-shifted form of these responses based on Eqn. S9 is shown illustration 1102 in FIG. 11A. White-noise responses were added with the same size for comparison, as shown in illustration 1103 in FIG. 11A. Performing SVD to the three sets of responses, observed are different compression efficiencies from the perspectives of normalized singular value as shown in FIG. 11B and proportion of the variance in FIG. 11C. In both FIGS. 11B and 11C, it can be seen that the compression efficiency of the original responses is similar to that of the white-noise responses, whereas the efficiency for the temporally-shifted responses is significantly higher and thus advantageous for the compression.

B. Point Source Response of an Ultrasonic Transducer with a Flat Rectangular Detection Surface

In this Section III(B), an example of a point source (virtual transducer) system response for a virtual transducer element of an ultrasonic transducer with a flat rectangular detection surface is discussed.

The forward operator described in Section III(A) can be configured for an ultrasonic transducer with any detection surface. In the example discussed in this Section III(B), the ultrasonic transducer has a flat rectangular detection surface. From Eqns. S6 and S15 it is shown that the forward operator is based on the point source response ĝ_{lcl}(r′_{lcl}, t) in the local coordinates. The response is determined by the first derivative of EIR (h′ₑ (t)) and the realigned SIR (ĥ_{s,lcl}(r′_{lcl}, t)) in the local coordinates. In general, ĥ_{lcl}(r′_{lcl}, t) can be measured experimentally. In this example, for an ultrasonic transducer with a flat rectangular detection surface, ĥ_{lcl}(r′_{lcl}, t) is calculated efficiently using the far-field approximation of ĥ_{s,lcl}(r′_{lcl}, t). From a special case of this approximation, a method is derived to measure ĥ_{lcl}(r′_{lcl},t) experimentally.

In the first step of obtaining ĥ_{lcl}(r′_{lcl}, t), the calculation of ĥ_{lcl}(r′_{lcl}, t) is introduced based on the far-field approximation of ĥ_{s,lcl}(r′_{lcl}, t). In this research, the image domain is within the far-field regions of all transducer elements. Using the far-field approximation, the SIR is expressed in the frequency domain as:

$${F}_{t} ( {h}_{s , n} ( {r}^{′} t ) ) ( f ) \approx \frac{ab exp ( - j 2 π f \frac{‖ {r}^{′} - {r}_{n} ‖}{c} )}{2 π c ‖ {r}^{′} - {r}_{n} ‖} sin \text{} c ( π f \frac{a \vert ( {r}^{′} - {r}_{n} ) {\hat{x}}_{n}^{T} \vert}{c ‖ {r}^{′} - {r}_{n} ‖} ) sin c ( π f \frac{b \vert ( {r}^{′} - {r}_{n} ) {\hat{y}}_{n}^{T} \vert}{c ‖ {r}^{′} - {r}_{n} ‖} ) . \qquad ( Eqn . S 19 )$$

Here, Fₜ denotes the temporal FT, and j denotes the imaginary unit. Combining Eqns. S14 and S19, the temporal Fourier transform (FT) is applied to ĥ_{s,lcl}(r′_{lcl}, t) to obtain:

$${F}_{t} ( {\hat{h}}_{s , lcl} ( {r}_{lcl}^{′} , t ) ) ( f ) = {F}_{t} ( {h}_{s , n} ( {r}^{′} , t + \frac{‖ {r}^{′} - {r}_{n} ‖}{c} ) ) ( f ) = exp ( j 2 π f \frac{‖ {r}^{′} - {r}_{n} ‖}{c} ) {F}_{t} ( {h}_{s , n} ( {r}^{′} , t ) ) ( f ) \approx \frac{a b}{2 π c ‖ {r}^{′} - {r}_{n} ‖} sin c ( π f \frac{a \vert ( {r}^{′} - {r}_{n} ) {\hat{x}}_{n}^{T} \vert}{c ‖ {r}^{′} - {r}_{n} ‖} ) sin c ( π f \frac{b \vert ( {r}^{′} - {r}_{n} ) {\hat{y}}_{n}^{T} \vert}{c ‖ {r}^{′} - {r}_{n} ‖} ) = \frac{a b}{2 π c ‖ {r}_{lcl}^{′} ‖} sin c ( π f \frac{a \vert {x}_{lcl}^{′} \vert}{c ‖ {r}_{lcl}^{′} ‖} ) sin c ( π f \frac{b \vert {y}_{lcl}^{′} \vert}{c ‖ {r}_{lcl}^{′} ‖} ) . \qquad ( Eqn . S20 )$$

Here, identities r′_{lcl}=(r′−rₙ)Aₙ=(r′−rₙ)(\hat{x}ₙ^{T}, ŷₙ^{T}, \hat{z}ₙ^{T})=(x′_{lcl}, y′_{lcl}, z′_{lcl}) and ∥r\hat{ } ′−r_n∥=∥r_lcl\hat{ }′∥, and r′_{lcl} must not be the origin in the local coordinates. Substituting Eqn. S20 into the temporal FT of Eqn. S13, obtains:

$${F}_{t} ( {\hat{h}}_{lcl} ( {r}_{lcl}^{′} , t ) ) ( f ) = \frac{1}{2 c a b} {F}_{t} ( {h}_{e}^{′} ( t ) ) ( f ) {F}_{t} ( {\hat{h}}_{s , lcl} ( {r}_{lcl}^{′} , t ) ) ( f ) \approx \frac{{F}_{t} ( {h}_{e}^{′} ( t ) ) ( f )}{4 π {c}^{2} ‖ {r}_{lcl}^{′} ‖} sin c ( π f \frac{a \vert {x}_{lcl}^{′} \vert}{c ‖ {r}_{lcl}^{′} ‖} ) sin c ( π f \frac{b \vert {y}_{lcl}^{′} \vert}{c ‖ {r}_{lcl}^{′} ‖} ) . \qquad ( Eqn . S21 )$$

Applying temporal inverse FT to Eqn. S21, obtains:

$${\hat{h}}_{lcl} ( {r}_{lcl}^{′} , t ) \approx {F}_{t}^{- 1} ( \frac{{F}_{t} ( {h}_{e}^{′} ( t ) ) ( f )}{4 π {c}^{2} ‖ {r}_{lcl}^{′} ‖} sin c ( π f \frac{a \vert {x}_{lcl}^{′} \vert}{c ‖ {r}_{lcl}^{′} ‖} ) sin c ( π f \frac{b \vert {y}_{lcl}^{′} \vert}{c ‖ {r}_{lcl}^{′} ‖} ) ) . \qquad ( Eqn . S22 )$$

In the second step of obtaining ĥ_{lcl}(r′_{lcl}, t), a method is derived to measure h′ₑ(t) by analyzing a special case of Eqn. S22. The location r′_{lcl} is constrained on the axis of the transducer by letting r′_{lcl}=(0, 0, z′_{lcl}), which simplifies Eqn. S22 to:

$${\hat{h}}_{lcl} ( {r}_{lcl}^{′} , t ) \approx {F}_{t}^{- 1} ( \frac{{F}_{t} ( {h}_{e}^{′} ( t ) ) ( f )}{4 π {c}^{2} \vert {z}_{lcl}^{′} \vert} ) = \frac{{h}_{e}^{′} ( t )}{4 π {c}^{2} \vert {z}_{lcl}^{′} \vert} . \qquad ( Eqn . S23 )$$

Solving for h′ₑ(t) from Eqn. S23, obtains:

$${h}_{e}^{′} ( t ) \approx 4 π {c}^{2} \vert {z}_{lcl}^{′} \vert {\hat{h}}_{lcl} ( {r}_{lcl}^{′} , t ) = 4 π {c}^{2} \vert {z}_{lcl}^{′} \vert {\hat{h}}_{n} ( {r}^{′} , t ) = 4 π {c}^{2} \vert {z}_{lcl}^{′} \vert {h}_{n} ( ( 0 , 0 , {z}_{lcl}^{′} ) {A}_{{r}_{n}}^{- 1} + {r}_{n} , t + \frac{\vert {z}_{lcl}^{′} \vert}{c} ) . \qquad ( Eqn . S24 )$$

Here, the identity r′=r′_{lcl}Aₙ⁻¹+rₙ=(0, 0, z_{lcl})Aₙ⁻¹+rₙ is used. In this example, the measurement of the right-hand side of Eqn. S24 is repeated and the average used to represent h′ₑ(t).

In summary, h′ₑ(t) is measured experimentally on the basis of Eqn. S24 and the measurement is substituted into Eqn. S22 to obtain ĥ_{lcl}(r′_{lcl}, t). Further, SVD is performed to ĥ_{lcl}(r′_{lcl}, t) according to Eqn. S16 and obtain singular functions ĥ_{lcl,k} (r′_{lcl}) and ηₖ(t), k=1, 2, . . . , K are obtained. These functions in Eqn. S18 are used to implement the fast forward operator.

### C. Discretization of the Forward Operator

In this subsection III(C), a fast forward operator for a TISMC method of an embodiment is discretized for use to determine values that can be used, for example, to determine coefficients used in the forward simulation.

In certain examples, a forward operator of a TISMC method may be expressed in two different forms as provided in Eqns. S6 and S18, respectively. First, the forward operator implemented in Eqn. S6 is discretized. In the temporal domain, points of interest tₗ=t₁+(l−1)τ, l=1, 2, . . . , L, are chosen where t₁ is the initial time, τ is the sampling step size, and L is the number of time points. Then, the temporal FT of h′ₑ(t) as Fₜ(h′ₑ(t))_{l′}≈F₁(h′ₗ)(l′), l′=1, 2, . . . , L, is discretized where h′ₗ=h′ₑ(tₗ), Fₜ(h′ₑ(t))_{l′}=F(h′ₑ(t))(f_{l′}), l, l′∈{1, 2, . . . , L}, and Fₗ represents the discrete FT with respect to l. The temporal frequencies f_{l′}, l′=1, 2, . . . , L are selected according to the requirement of the discrete FT. Further, the m_{lcl}-th location in the local coordinates is denoted as r′_{lcl,m}_{_{lcl}}=(x′_{lcl,m}_{_{lcl}}, y′_{lcl,m}_{_{lcl}}, z′_{lcl,m}_{_{lcl}}) and ĥ_{lcl,m}_{_{lcl}}ₗ=ĥ_{lcl}(r′_{lcl,m}_{_{lcl}}, tₗ), m_{lcl}=1, 2, . . . , M_{lcl}, is defined where M_{lcl} is the number of locations of interest in the local coordinates. Thus, Eqn. S22 is discretized as:

$${\hat{h}}_{lcl , {m}_{lcl} , l} \approx \frac{1}{4 π {c}^{2} ‖ {r}_{lcl , {m}_{lcl}}^{′} ‖} {F}_{l}^{- 1} ( {F}_{l} ( {h}_{l}^{′} ) ( {l}^{′} ) sin c ( π {f}_{{l}^{′}} \frac{a \vert {x}_{lcl , {m}_{lcl}}^{′} \vert}{c ‖ {r}_{lcl , {m}_{lcl}}^{′} ‖} ) sin c ( π {f}_{{l}^{′}} \frac{b \vert {y}_{lcl , {m}_{lcl}}^{′} \vert}{c ‖ {r}_{lcl , {m}_{lcl}}^{′} ‖} ) ) ( l ) , \\ {m}_{lcl} = 1 , 2 , … , {M}_{lcl} , l = 1 , 2 , … , L . \qquad ( Eqn . S25 )$$

Further, the point source response hₙ(r′, t) are discretized as h_{n,m,l}=hₙ(r′ₘ, tₗ), n=1, 2, . . . , N, m=1, 2, . . . , M, l=1, 2, . . . , L. Here, M is the number of voxels (source points) in the image domain. On the basis of the relation

$${h}_{n} ( {r}_{m}^{′} , {t}_{l} ) = {\hat{h}}_{n} ( {r}_{m}^{′} , {t}_{l} - \frac{‖ {r}_{m}^{′} - {r}_{n} ‖}{c} ) = {\hat{h}}_{lcl} ( ( {r}_{m}^{′} - {r}_{n} ) {A}_{n} , {t}_{l} - \frac{‖ {r}_{m}^{′} - {r}_{n} ‖}{c} ) ,$$

the values of h_{n,m,l} are obtained through spatiotemporal interpolation of the values of ĥ_{lcl,m}_{_{lcl}}_{,l}. Denoting \hat{p}_{n,l}=\hat{p}(rₙ, tₗ) and p_{0,m}=p₀(r′ₘ), Eqn. S6 is discretized as:

$${\hat{p}}_{n , l}^{slow} = {\sum }_{m = 1}^{M} {v}_{m} {h}_{n , m , l} {p}_{0 , m} , n = 1 , 2 , … , N , l = 1 , 2 , … , L . \qquad ( Eqn . S26 )$$

Here, \hat{p}_{n,l}^{slow} represents an approximation of \hat{p}_{n,l} using this relatively slow operator, and vₘ represents the volume of the m-th voxel. In this example, due to the finite duration of every point source response shown in FIGS. 10A, in illustration 1101 of FIG. 11A, and in illustration 1102 of FIG. 11A, given a combination of m and n, it is needed to calculate only for l in a range of length L′<L. Here, L′ is the effective length for nonzero values in the discretized point source responses, and the computational complexity of the discrete forward operator in Eqn. S26 is O(NML′).

Next, the forward operator is discretized to a form with lower computational complexity from Eqn. S18. h̅_{lcl,k,m}_{_{lcl}}=ĥ_{lcl,k}(r′_{lcl,m}_{_{lcl}}) and η_{k,l}=ηₖ(tₗ), k=1, 2, . . . , K, m_{lcl}=1, 2, . . . , M_{lcl}, l=1, 2, . . . , L. After obtaining the array ĥ_{lcl,m}_{_{lcl}}_{,l} through Eqn. S25, ĥ_{lcl,k,m}_{_{lcl}} and η_{k,l} are estimated through SVD:

$${\hat{h}}_{lcl , {m}_{lcl} , l} \approx {\sum }_{k = 1}^{K} {\overline{h}}_{lcl , k , {m}_{lcl}} {η}_{k , l} , {m}_{lcl} = 1 , 2 , … , {M}_{lcl} , l = 1 , 2 , … , L . \qquad ( Eqn . S27 )$$

ĥ_{lcl,k,n,m}=ĥ_{lcl,k}((r′ₘ−rₙ)Aₙ), which is obtained from the values of ĥ_{lcl,k,m}_{_{lcl}} through spatial interpolation. Moreover,

$$δ ( t - \frac{‖ {r}^{′} - {r}_{n} ‖}{c} )$$

is expressed in the discrete form as:

$$\frac{1}{τ} ( ( {t}_{{l}_{n , m} + 1} - \frac{‖ {r}_{m}^{′} - {r}_{n} ‖}{c} ) {δ}_{l , {l}_{n , m}} + ( \frac{‖ {r}_{m}^{′} - {r}_{n} ‖}{c} - {t}_{{l}_{n , m}} ) {δ}_{l , {l}_{n , m} + 1} ) , \\ n = 1 , 2 , … , N , m = 1 , 2 , … , M , l = 1 , 2 , … , L . \qquad ( Eqn . S28 )$$

Here, l_{n,m} denotes the temporal index such that

$${t}_{{l}_{n , m}} \leq \frac{‖ {r}_{m}^{′} - {r}_{n} ‖}{c} < {t}_{{l}_{n , m} + 1} ,$$

and the Kronecker delta function is used:

$${δ}_{l , {l}^{′}} = \{ \begin{matrix}0 , l \neq {l}^{′} , \\ 1 , l = {l}^{′}\end{matrix} . \qquad ( Eqn . S29 )$$

The forward operator in Eqn. S18 is discretized as:

$${\hat{p}}_{n , l}^{fast} = {\sum }_{k = 1}^{K} {η}_{k , {l}^{*} l} {\sum }_{m = 1}^{M} {v}_{m} {\hat{h}}_{lcl , k , n , m} {p}_{0 , m} \frac{1}{τ} [ \begin{matrix}( {t}_{{l}_{n , m} + 1} - \frac{‖ {r}_{m}^{′} - {r}_{n} ‖}{c} ) {δ}_{l , {l}_{n , m}} + \\ ( \frac{‖ {r}_{m}^{′} - {r}_{n} ‖}{c} - {t}_{{l}_{n , m}} ) {δ}_{l , {l}_{n , m} + 1}\end{matrix} ] , \\ n = 1 , 2 , … , N , l = 1 , 2 , … , L . \qquad ( Eqn . S30 )$$

Here, \hat{p}_{n,l}^{fast} represents an approximation of \hat{p}_{n,l} using the fast operator, and *ₗ denotes the discrete convolution with respect to l. In this example, a value of L is chosen so that log₂ L is an integer, and the discrete convolution is implemented using the temporal fast Fourier transform (FFT). Thus, the computational complexity of the discrete forward operator in Eqn. S30 is O(NMK)+O(N(L log₂ L)K).

### D. Efficiency and Signal-Domain Accuracy of the Fast Forward Operator

In this Section III(D), the efficiency and signal-domain accuracy of a fast forward operator for an example of a TISMC method according to an embodiment is discussed.

To quantify the efficiency and accuracy of a fast forward operator for the TISMC method determined based on Eqn. S30, numerical simulations were performed by placing a numerical phantom of size 1×1×1 cm³ in four image subdomains D₁, D₂, D₃, and D₄ of a PACT system, according to an embodiment. FIG. 12A depicts an illustration of the numerical phantom formed by three rectangular cuboids (each of size 2.6×2.6×10 mm³) intersecting at their centers, according to an implementation. Voxel values are 1 inside the phantom and 0 outside. Three lines (L1, L2, and L3) are picked for image-domain comparisons shown in FIG. 14. FIG. 12B depicts a virtual 2D array (solid arcs), four subdomains D₁, D₂, D₃, and D₄, each of size 1×1×1 cm³, and the domain occupied by rotations of the four subdomains around the array axis (dotted circles and arcs), according to an implementation.

The virtual 2D array is formed by the rotation of the four arc arrays is marked by arcs and the rotation of the four subdomains around the same axis covers a domain (marked by dotted circles and arcs) enclosing all the image domains. A slow operator is implemented according to Eqn. S26 for comparison.

FIG. 12C depicts a plot of computation times (t_{fast} and t_{slow}) of the fast and slow operators, respectively, for the forward simulations with the numerical phantom in D₁ (36 repetitions), according to an implementation. FIG. 13A depicts images of relative errors of the simulated signals for all virtual elements (n′_{loc}=1, 2, . . . , 396, n′ₑₗₑ=1, 2, . . . , 128), with the numerical phantom in D₁, D₂, D₃, and D₄, according to an implementation. FIG. 13B depicts a comparison of the signals (\hat{p}^{slow} and \hat{p}^{fast}) simulated by the fast and slow operators for the 64-th element (n′ₑₗₑ=64) in the first virtual arc array (n′_{loc}=1), with the phantom in D₁, D₂, D₃, and D₄, respectively, in FIG. 13A.

The efficiency of the fast operator is quantified by performing forward simulations with the numerical phantom in the subdomain D₁ using both operators with parameters M=50×50×50 (voxel size of 0.2×0.2×0.2 mm³), N=396×128 (the four-arc array rotated for 99 locations and each arc downsized to 128 elements), L=4096, L′=151, and K=3. The single-thread-CPU version of each operator was run 36 times on a server with Ubuntu 20.04.6 LTS and Intel® Xeon® Gold 6248R CPU @ 3.00 GHz. Denoting the computation times of both operators as t_{fast} and t_{slow}, respectively, mean(t_{fast})=6.3 min, std(t_{fast})=0.4 min, mean(t_{slow})=267 min, and std(t_{slow})=19 min was obtained for the 36 simulations. A comparison of these values is shown in FIG. 12C. A Welch's t-test was performed between 42t_{fast} and t_{slow}, showing insignificant difference (p-value=0.59). Thus, the fast operator with K=3 has approximately 42 times the speed of the slow operator, and the acceleration ratio can be further improved by reducing K.

Then, the accuracy of the fast operator is demonstrated by performing forward simulations for the numerical phantom at the four subdomains (all the other parameters are the same as above). The results from the slow operator are regarded as ground truth (\hat{p}_{gt,n,l}=\hat{p}_{n,l}^{slow}) and the relative error of the fast-operator results (\hat{p}_{n,l}^{fast}) is defined as

$${e}_{n} = \frac{\sqrt{{\sum }_{l = 1}^{L} {( {\hat{p}}_{n , l}^{fast} - {\hat{p}}_{gt , n , l} )}^{2}}}{\sqrt{{\sum }_{l = 1}^{L} {\hat{p}}_{gt , n , l}^{2}}} ,$$

n=1, 2, . . . , N, l=1, 2, . . . , L. The values of relative error eₙ for the four subdomains (D₁, D₂, D₃, and D₄) are compared in FIG. 14, with n′_{loc} the index of the virtual arc array and n′ₑₗₑ the index of the element in each array, n′_{loc}=1, 2, . . . , 396, n′ₑₗₑ=1, 2, . . . , 128. These relative errors are generally small and all of them are below 0.005, which is enough for this study. For the 64-th element (n′ₑₗₑ=64) on the first virtual array (n′_{loc}=1), the signals generated by the slow and fast operators were compared, denoted as \hat{p}_{slow} and \hat{p}_{fast} respectively, which show the fast operator's high accuracy across the time domain.

FIG. 14 are plots of the relative error of the reconstructed image in subdomains D₁, D₂, D₃, and D₄ with 1 to 256 iterations, values on the lines L1, L2, and L3 in the reconstructed images with nᵢₜₑᵣ=1, 4, 8, 64, 256, and a comparison between ground truth (p_{0,L1}, p_{0,L2}, and p_{0,L3}) and 256-iteration reconstructed (\hat{p}_{0,L1}, \hat{p}_{0,L2}, and \hat{p}_{0,L3}) values along the three lines, according to an implementation.

### E. Regularized Iterative Reconstruction and Image-Domain Accuracy of the Fast Forward Operator of TISMC Methods

In this Section III(E), the image-domain accuracy of a fast forward operator for an example of a TISMC method according to an embodiment is discussed.

The discretized initial pressure and transducer response is denoted as column vectors p₀ and \hat{p}, respectively, and the forward model is expressed as:

$$\\ \begin{matrix}\hat{p} = {Hp}_{0} , & ( Eqn . S 31 )\end{matrix}$$

where H is the discretized system matrix of shape (NL, M). If H is full column rank (columns of the matrix are linearly independent), an image may be reconstructed by solving the optimization problem:

$$\\ \begin{matrix}{\hat{p}}_{0} = {arg min}_{{p}_{0} ∈ {ℝ}^{M}} {‖ {Hp}_{0} - \hat{p} ‖}^{2} & ( Eqn . S 32 )\end{matrix}$$

using FISTA with a constant step size, according to an embodiment.

The Lipschitz constant (the largest eigenvalue) of 2H^{T}H may be obtained through the power method with, e.g., 32 iterations and it may be used in FISTA. Further, nonnegativity of the initial pressure may be added as a constraint to facilitate the reconstruction:

$$\\ \begin{matrix}{\hat{p}}_{0} = {arg min}_{{p}_{0} ∈ {ℝ}^{M} , {p}_{0} \geq 0} {‖ {Hp}_{0} - \hat{p} ‖}^{2} . & ( Eqn . S 33 )\end{matrix}$$

If H is not full column rank or the noise level is high, the image may be reconstructed by solving the regularized optimization problem:

$$\\ \begin{matrix}{\hat{p}}_{0} = {arg min}_{{p}_{0} ∈ {ℝ}^{M} , {p}_{0} \geq 0} {γ}_{c} {‖ {Hp}_{0} - \hat{p} ‖}^{2} + λ {\vert {p}_{0} \vert}_{TV} & ( Eqn . S 34 )\end{matrix}$$

using FISTA with a constant step size. Here, A denotes the regularization parameter and ye represents the system-specific measurement calibration factor. In one example, instead of dealing with \hat{p} directly, γ_{c}\hat{p}₀ from γ_{c}\hat{p} (reading of the data acquisition system) are obtained by solving the problem

$$\\ {γ}_{c} {\hat{p}}_{0} = {arg min}_{{γ}_{c} {p}_{0} ∈ {ℝ}^{M} , {γ}_{c} {p}_{0} \geq 0} {‖ H ( {γ}_{c} {p}_{0} ) - {γ}_{c} \hat{p} ‖}^{2} + λ {\vert {γ}_{c} {p}_{0} \vert}_{TV} ,$$

which is equivalent to Eqn. S34. The normalized values of γ_{c}\hat{p}₀ (the same as those of p₀) are used for further analysis, and effect of γ_{c} is ignored in this example. The term |p₀|_{TV} in Eqn. S34 denotes the total variation (TV) norm, defined as:

$$\\ \begin{matrix}{\vert {p}_{0} \vert}_{TV} = {\sum }_{\begin{matrix}2 \leq {m}_{1} \leq {M}_{1} \\ 2 \leq {m}_{2} \leq {M}_{2} \\ 2 \leq {m}_{3} \leq {M}_{3}\end{matrix}} \sqrt{\begin{matrix}{( \frac{{p}_{0 , {m}_{1} , {m}_{2} , {m}_{3}} - {p}_{0 , {m}_{1} - 1 , {m}_{2} , {m}_{3}}}{{v}_{m , 1}} )}^{2} + \\ {( \frac{{p}_{0 , {m}_{1} , {m}_{2} , {m}_{3}} - {p}_{0 , {m}_{1} , {m}_{2} - 1 , {m}_{3}}}{{v}_{m , 2}} )}^{2} + \\ {( \frac{{p}_{0 , {m}_{1} , {m}_{2} , {m}_{3}} - {p}_{0 , {m}_{1} , {m}_{2} , {m}_{3} - 1}}{{v}_{m , 3}} )}^{2}\end{matrix}} . & ( Eqn . S 35 )\end{matrix}$$

In this definition, v_{m,1}, v_{m,2}, and v_{m,3} represent the voxel sizes along the x-axis, y-axis, and z-axis, respectively; M₁, M₂, and M₃ (M=M₁M₂M₃) denote the number of voxels in the first, second, and third dimensions, respectively; and the column vector p₀ to a 3D array p_{0,M}_{₁}_{×M}_{₂}_{×M}_{₃} is reshaped to express 3D information. Given a set of parameters for FISTA, the solution to Eqn. S32 is linearly dependent on \hat{p}. The solutions to Eqns. S33 and S34 are nonlinearly dependent on \hat{p} due to the nonnegative constraint (p₀≥0) and the TV regularization. Symbols for the forward operator and image reconstruction are summarized in Table 1.

The accuracy of the fast forward operator may be quantified in the image domain by performing forward simulations using the slow operator (H implemented through Eqn. S26, regarded as ground truth) and image reconstructions using iterations with nonnegativity constraints (Eqn. S33 with H implemented through Eqn. S30 and accelerated by two NVIDIA A100 GPUs), with the numerical phantom shown in FIG. 12A in the four subdomains D₁, D₂, D₃, and D₄, respectively. The reconstructed image \hat{p}₀'s relative error to the ground-truth image p_{0,gt} is defined as

$$\\ \frac{‖ {\hat{p}}_{0} - {p}_{0 , gt} ‖}{‖ {p}_{0 , gt} ‖} .$$

For the reconstructions with the numerical phantom in D₁, the relative error reduces to 0.003 as the iteration number nᵢₜₑᵣ reaches 256, as shown in FIG. 14. In FIG. 14, the values of the reconstructed images are plotted along three lines (L1, L2, and L3) for nᵢₜₑᵣ=1, 4, 8, 64, 256. Further, the ground-truth (256-iteration reconstructed) values along the lines are denoted as p_{0,L1}, p_{0,L2}, and p_{0,L3} (\hat{p}_{0,L1}, \hat{p}_{0,L2}, and \hat{p}_{0,L3}), respectively, and compared in FIG. 14. All the plots show the high accuracy of the fast forward operator in the image domain.

### F. Intra-Image Nonrigid Motion Correction

Certain embodiments pertain to motion correction methods that can be implemented with a PACT system. In these cases, a transducer array with Nₑₗₑ elements may be moved across N_{loc} locations to form a virtual 2D transducer array for 3D imaging (with the number of transducer elements N=N_{loc}Nₑₗₑ). The motion of a source point in the image domain corresponds to temporal shifts of the signals from the source point in the signal domain. To encode the motion information in the forward operator, Eqn. S18 is modified to:

$$\\ \begin{matrix}{\hat{p}}_{ζ} ( {r}_{{n}_{loc} , {n}_{ele}} , t ) \approx {\sum }_{k = 1}^{K} {η}_{k} ( t ) {*}_{t} {\int }_{V} {p}_{0} ( {r}^{{}_{} ′} ) {\hat{h}}_{lcl , k} ( ( {r}^{{}_{} ′} - {r}_{{n}_{loc} , {n}_{ele}} ) {A}_{{r}_{{n}_{loc} , {n}_{ele}}} ) {}_{} δ {}_{} ( t - ζ ( {r}^{{}_{} ′} , {r}_{{n}_{loc} , {n}_{ele}} ) - \frac{‖ {r}^{{}_{} ′} - {r}_{{n}_{loc} , {n}_{ele}} ‖}{c} ) {dr}^{{}_{} ′} , & ( Eqn . S 36 )\end{matrix}$$

$$\\ {n}_{loc} = 1 , 2 , … , {N}_{loc} , {n}_{ele} = 1 , 2 , … , {N}_{ele} , t \geq 0.$$

Here rₙ_{_{loc}}_{,n}_{ₑₗₑ}=rₙ_{_{loc}}+(nₑₗₑ−1)N_{loc} and ξ(r′, rₙ_{_{loc}}_{,n}_{ₑₑₗ}) is defined to be the motion-induced temporal shift of the signals from the source point at r′ that are detected by the nₑₗₑ-th element when the transducer array is at the n_{loc}-th location. Due to motion, a source point does not stay at r′, and r′ is used to represent the source point's average location.

Next, the temporal shifts ξ(r′, rₙ_{_{loc}}_{,n}_{ₑₗₑ}) are connected with the motions. For efficiency and robustness, instead of modeling the motions of all source points in the image domain D, the motions of subdomains D₁, D₂, . . . , D_{M}_{_{sub}} occupying the image domain are modeled as:

$$\\ \begin{matrix}D = {D}_{1} ⋃ {D}_{2} ⋃ … ⋃ {D}_{{M}_{sub}} . & ( Eqn . S 37 )\end{matrix}$$

For simplicity, all subdomains are set to be rectangular cuboids of the same size. The center of the m_{sub}-th subdomain Dₘ_{_{sub}} is denoted as r′_{c,m}_{_{sub}} and the motions of r_{c,m}_{_{sub}} are used to represent the motions of Dₘ_{_{sub}}, m_{sub}=1, 2, . . . , M_{sub}.

Generally, the motions of tissues are small during data acquisition, and the deformations and rotations of each subdomain are small. Because of this, the subdomains may be considered rigid and the deformations and rotations of each subdomain ignored. The translations may be discretized with step sizes aₓ, a_{y}, and a_{z} along the x-axis, y-axis, and z-axis, respectively. The motion-induced translation steps of the domain center from r′_{c,m}_{_{sub}} are represented as kₓ(r′_{c,m}_{_{sub}}, n_{loc}), k_{y}(r′_{c,m}_{_{sub}}, n_{loc}), and k_{z}(r′_{c,m}_{_{sub}}, n_{loc}) along the x-axis, y-axis, and z-axis, respectively, when the transducer array is at the n_{loc}-th location. In summary, the temporal shifts caused by these translations are expressed as:

$$\\ \begin{matrix}{ζ}_{k} ( {r}_{c , {m}_{sub}}^{{}_{} ′} , {r}_{{n}_{loc} , {n}_{ele}} ) = \frac{‖ {r}_{c , {m}_{sub}}^{{}_{} ′} + k ( {r}_{c , {m}_{sub}}^{{}_{} ′} , {n}_{loc} ) ⊙ a - {r}_{{n}_{loc} , {n}_{ele}} ‖}{c} - \frac{‖ {r}_{c , {m}_{sub}}^{{}_{} ′} - {r}_{{n}_{loc} , {n}_{ele}} ‖}{c} , & ( Eqn . S 38 )\end{matrix}$$

$$\\ {m}_{sub} = 1 , 2 , … , {M}_{sub} , {n}_{loc} = 1 , 2 , … , {N}_{loc} , {n}_{ele} = 1 , 2 , … , {N}_{ele} .$$

Here, a constant vector a=(aₓ, a_{y}, a_{z}) and variable vectors k(r′_{c,m}_{_{sub}}, n_{loc}) (kₓ(r′_{c,m}_{_{sub}}, n_{loc}), k_{y} (r′_{c,m}_{_{sub}}, n_{loc}), k_{z}(r′_{c,m}_{_{sub}}, n_{loc}))∈ [symbol] ³, the variable vectors are grouped into a set k={k(r′_{c,m}_{_{sub}}, n_{loc})∈ [symbol] ³|m_{sub}=1, 2, . . . , M_{sub}, n_{loc}=1, 2, . . . , N_{loc}}. Then, the values of ξ(r′, r′ₙ_{_{loc}}_{,n}_{ₑₗₑ}) are obtained from the values of ξₖ(r′_{c,m}_{_{sub}} rₙ_{_{loc}}_{,n}_{ₑₗₑ}) through spatial interpolation in the image domain, which has high accuracy due to the spatial smoothness of the motions of tissues. Thus, the temporal shifts ξ(r′, rₙ_{_{loc}}_{,n}_{ₑₗₑ}) are connected with the motions, described by k.

Furthermore, the motion-incorporated forward operator in Eqn. S36 may be discretized and a motion correction method may be determined through Gauss-Seidel-type iterations. ξ(r′, rₙ_{_{loc}}_{,n}_{ₑₗₑ}) is replaced with ξk(r′, rₙ_{_{loc}}_{,n}_{ₑₗₑ}) in Eqn. S36 to emphasize the temporal shifts' dependency on k. Then, the forward operator is discretized to:

$$\\ \begin{matrix}{\hat{p}}_{k , n , l} \approx {\sum }_{k = 1}^{K} {η}_{k , l} {*}_{l} {\sum }_{m = 1}^{M} {v}_{m} {\hat{h}}_{lcl , k , n , m} {p}_{0 , m} \frac{1}{τ} [ \begin{matrix}( {t}_{{l}_{n , m} + 1} - {ζ}_{k} ( {r}_{m}^{{}_{} ′} , {r}_{n} ) - \frac{‖ {r}_{m}^{{}_{} ′} - {r}_{n} ‖}{c} ) {}_{} {δ}_{l , {l}_{n , m}} + \\ ( {ζ}_{k} ( {r}_{m}^{{}_{} ′} , {r}_{n} ) + \frac{‖ {r}_{m}^{{}_{} ′} - {r}_{n} ‖}{c} - {t}_{{l}_{n , m}} ) {}_{} {δ}_{l , {l}_{n , m} + 1}\end{matrix} ] , & ( Eqn . S 39 )\end{matrix}$$

$$\\ n = 1 , 2 , … , N , l = 1 , 2 , … , L ,$$

where, l_{n,m} denotes the temporal index such that

$$\\ {t}_{{l}_{n , m}} \leq {ζ}_{k} ( {r}_{m}^{{}_{} ′} , {r}_{n} ) + \frac{‖ {r}_{m}^{{}_{} ′} - {r}_{n} ‖}{c} < {t}_{{l}_{n , m} + 1} .$$

Accordingly, the forward operator is expressed in matrix form as:

$$\\ \begin{matrix}\hat{p} = {H}_{k} {p}_{0} , & ( Eqn . S 40 )\end{matrix}$$

through which the data-driven motion correction is expressed as a dual-objective optimization problem:

$$\\ \begin{matrix}( {\hat{p}}_{0} , \hat{k} ) = {arg min}_{{p}_{0} ∈ {ℝ}^{M} , {p}_{0} \geq 0 , k ∈ {( {ℤ}^{3} )}^{{M}_{sub} {N}_{loc}}} {γ}_{c} {‖ {H}_{k} {p}_{0} - \hat{p} ‖}^{2} + λ {\vert {p}_{0} \vert}_{TV} . & ( Eqn . S 41 )\end{matrix}$$

This is simplified into a convex optimization problem and a combinatorial optimization problem. The convex optimization problem, may be expressed as Eqn. S42 below, which can be solved by FISTA:

$$\\ \begin{matrix}{\hat{p}}_{0} = {arg min}_{{p}_{0} ∈ {ℝ}^{M} , {p}_{0} \geq 0} {γ}_{c} {‖ {H}_{k} {p}_{0} - \hat{p} ‖}^{2} + λ {\vert {p}_{0} \vert}_{TV} , & ( Eqn . S 42 )\end{matrix}$$

The combinatorial optimization problem may be expressed as:

$$\hat{k} = {arg min}_{k ∈ {( {ℤ}^{3} )}^{M} {sub}^{N} loc} {‖ {H}_{k} {p}_{0} - \hat{p} ‖}^{2} , \qquad ( Eqn . S43 )$$

Instead of solving the problem in Eqn. S43 directly, each vector k(r′_{c,m}_{_{sub}}, n_{loc}) may be solved independently without changing other vectors:

$$\hat{k} ( {r}_{c , {m}_{sub}}^{′} , {n}_{loc} ) = {arg min}_{k ( {r}_{c , {m}_{sub}}^{′} , {n}_{loc} ) ∈ {ℤ}^{3}} {‖ {H}_{k} {p}_{0} - \hat{p} ‖}^{2} , \qquad ( Eqn . S44 )$$

$${m}_{sub} = 1 , 2 , … , {M}_{sub ,}$$

$${n}_{loc} = 1 , 2 , … , {N}_{loc} .$$

To further simplify Eqn. S44, for each subdomain Dᵢ, p₀ may be decomposed into two images:

$${p}_{0} = {p}_{0 , {D}_{{m}_{sub}}} + {p}_{0 , D ∖ {D}_{{m}_{sub}}} , \qquad ( Eqn . S45 )$$

$${m}_{sub} = 1 , 2 , … , {M}_{sub} .$$

Here

$${p}_{0 , {D}_{{m}_{sub}}}$$

is zero outside the subdomain Dₘ_{_{sub}} and the same as p₀ inside Dₘ_{_{sub}}, whereas

$${p}_{0 , D ∖ {D}_{{m}_{sub}}}$$

is zero inside the subdomain Dₘ_{_{sub}} and the same as p₀ outside Dₘ_{_{sub}}. Using this decomposition, Eqn. S44 may be rewritten as:

$$( Eqn . S46 )$$

$$\begin{matrix}\hat{k} ( {r}_{c , {m}_{s u b}}^{′} , {n}_{loc} ) = {arg min}_{k ( {r}_{c , {m}_{s u b}}^{′} , {n}_{loc} ) ∈ {ℤ}^{3}} {‖ {H}_{k} ( {p}_{0 , {D}_{{m}_{sub}}} + {p}_{0 , D ∖ {D}_{{m}_{sub}}} ) - \hat{p} ‖}^{2} \\ = {arg min}_{k ( {r}_{c , {m}_{s u b}}^{′} , {n}_{loc} ) ∈ {ℤ}^{3}} \{ \begin{matrix}{‖ {H}_{k} ( {p}_{0 , {D}_{{m}_{sub}}} - \hat{p} ‖}^{2} + 2 {p}_{0 , {D}_{{m}_{sub}}}^{T} \\ {H}_{k}^{T} {H}_{k} {p}_{0 , D ∖ {D}_{{m}_{sub}}} + \\ {‖ {H}_{k} {p}_{0 , D ∖ {D}_{{m}_{sub}}} ‖}^{2} - 2 {\hat{p}}^{T} {H}_{k} {p}_{0 , D ∖ {D}_{{m}_{sub}}}\end{matrix} \} ,\end{matrix}$$

$${m}_{sub} = 1 , 2 , … , {M}_{sub} ,$$

$${n}_{loc} = 1 , 2 , … , {N}_{loc} .$$

Given the initial pressure

$${p}_{0 , D ∖ {D}_{{m}_{sub}}} ,$$

forward simulation (Hₖ) may be applied and adjoint reconstruction (Hₖ^{T}) may be applied to it to obtain

$${H}_{k}^{T} {H}_{k} {p}_{0 , D ∖ {D}_{{m}_{sub}}} ,$$

which has small amplitudes inside Dₘ_{_{sub}} for the detection geometry used by the PACT system. Thus,

$$2 {p}_{0 , {D}_{{m}_{sub}}}^{T} {H}_{k}^{T} {H}_{k} {p}_{0 , D ∖ {D}_{{m}_{sub}}} \approx 0.$$

Additionally, although the values of k(r′_{c,m}_{_{sub}}, n_{loc}) (the motion inside Dₘ_{_{sub}}) affect

$${H}_{k} {p}_{0 , D ∖ {D}_{{m}_{sub}}}$$

(the signals from the sources outside Dₘ_{_{sub}}) due to spatial interpolation, these effects are minor compared with the values' effects on

$${H}_{k} {p}_{0 , {D}_{{m}_{sub}}} .$$

### Thus,

$${‖ {H}_{k} {p}_{0 , D ∖ {D}_{{m}_{sub}}} ‖}^{2}$$

$$and$$

$$2 {\hat{p}}^{T} {H}_{k} {p}_{0 , D ∖ {D}_{{m}_{sub}}}$$

may be ignored in the optimization for k(r′_{c,m}_{_{sub}}, n_{loc}). Through these approximations, Eqn. S46 may be simplified to:

$$\begin{matrix}\hat{k} ( {r}_{c , {m}_{sub}}^{′} , {n}_{loc} ) \approx {arg min}_{k ( {r}_{c , {m}_{s u b}}^{′} , {n}_{loc} ) ∈ {ℤ}^{3}} {‖ {H}_{k} {p}_{0 , {D}_{{m}_{sub}}} - \hat{p} ‖}^{2} \\ = {arg min}_{k ( {r}_{c , {m}_{s u b}}^{′} , {n}_{loc} ) ∈ {ℤ}^{3}} {‖ {H}_{k , {n}_{loc}} {p}_{0 , {D}_{{m}_{sub}}} - {\hat{p}}_{{n}_{loc}} ‖}^{2} ,\end{matrix} \qquad ( Eqn . S47 )$$

$${m}_{sub} = 1 , 2 , … , {M}_{sub} ,$$

$${n}_{loc} = 1 , 2 , … , {N}_{loc} .$$

Here, \hat{p}n_{loc} (of shape NₑₗₑL×1) denotes the signals detected by the transducer array when it is at the n_{loc}-th location whereas signals for other locations are not affected by k(r′_{c,m}_{_{sub}}, n_{loc}), and H_{k,n}_{_{loc}}(of shape NₑₗₑL×M) denotes the corresponding system matrix slices. Additionally, the forward operator allows for forward simulation of any subdomain Dₘ_{_{sub}}(with MSD voxels), which is much smaller than the whole image domain D (with M voxels). Hence, obtaining \hat{k}(r′_{c,m}_{_{sub}}n_{loc}) using Eqn. S47 is much more efficient than obtaining it through Eqn. S44. The optimization problems in Eqns. S42 and S47 may be solved in multiple iterations in some cases. Thus, instead of obtaining an accurate choice of k(r′_{c,m}_{_{sub}}, n_{loc}) in each iteration, the optimization problem in Eqn. S47 may be further decomposed into problems:

$${\hat{k}}_{x} ( {r}_{c , {m}_{sub}}^{′} , {n}_{loc} ) = {arg min}_{{k}_{x} ( {r}_{c , {m}_{sub}}^{′} , {n}_{loc} ) ∈ \{ - {K}_{x ,} - {K}_{x} + 1 , … , {K}_{x} \}} {‖ {H}_{k , {n}_{loc}} {p}_{0 , {D}_{{m}_{sub}}} - {\hat{p}}_{{n}_{loc}} ‖}^{2} , \qquad ( Eqn . S48 )$$

$${\hat{k}}_{y} ( {r}_{c , {m}_{sub}}^{′} , {n}_{loc} ) = {arg min}_{{k}_{y} ( {r}_{c , {m}_{sub}}^{′} , {n}_{loc} ) ∈ \{ - {K}_{y ,} - {K}_{y} + 1 , … , {K}_{y} \}} {‖ {H}_{k , {n}_{loc}} {p}_{0 , {D}_{{m}_{sub}}} - {\hat{p}}_{{n}_{loc}} ‖}^{2} , \qquad ( Eqn . S49 )$$

$$and$$

$${\hat{k}}_{z} ( {r}_{c , {m}_{sub}}^{′} , {n}_{loc} ) = {arg min}_{{k}_{z} ( {r}_{c , {m}_{sub}}^{′} , {n}_{loc} ) ∈ \{ - {K}_{z ,} - {K}_{z} + 1 , … , {K}_{z} \}} {‖ {H}_{k , {n}_{loc}} {p}_{0 , {D}_{{m}_{sub}}} - {\hat{p}}_{{n}_{loc}} ‖}^{2} , \qquad ( Eqn . S50 )$$

$${m}_{sub} = 1 , 2 , … , {M}_{sub} ,$$

$${n}_{loc} = 1 , 2 , … , {N}_{loc} ,$$

with limited searching ranges determined by parameters Kₓ, K_{y}, and K_{z}, respectively. For each subdomain and each transducer array location, the problems in Eqns. S48-S50 may be solved by performing the forward simulation for the subdomain and the transducer array location for 2Kₓ+1, 2K_{y}, and 2K_{y} times, respectively. In summary, the computational complexity of updating all values in k once is:

$$( Eqn . S51 )$$

$${N}_{loc} {M}_{sub} [ 2 ( {K}_{x} + {K}_{y} + {K}_{z} ) + 1 ] [ O ( {N}_{ele} {M}_{SD} K ) + O ( {N}_{ele} ( L {log}_{2} L ) K ) ] .$$

FIG. 15 depicts a flowchart of a workflow of an example of a motion correction method, according to an embodiment. In addition, FIG. 4 depicts a flowchart of an example of a motion correction method, according to an embodiment. Additional symbols used in various examples of the motion correction method are provided in Table 2.

TABLE 2
Additional symbols reference in examples of intra-image nonrigid motion correction.

| Symbol | Definition | Example Values |
|---|---|---|
| I_{cor} (i_{cor}) | The number of motion | I_{cor} = 16 for numerical simulations |
| | corrections (index) | and in vivo experiments |
| M_{sub} (m_{sub}) | The number of image | M_{sub} = 3 × 3 × 2 for numerical |
| | subdomains for motion | simulations |
| | correction (index) | M_{sub} = 6 × 6 × 4 for in vivo |
| | | experiments |
| αₓ, α_{y}, and α_{z} (m) | Translation step sizes of | (αₓ, α_{y}, α_{z}) = (0.1, 0.1, 0.1) (mm) |
| | each subdomain along the x- | for numerical simulations and in |
| | axis, y-axis, and z-axis, | vivo experiments |
| | respectively | |
| Kₓ, K_{y}, and K_{z} | Searching step ranges along | (Kₓ, K_{y}, K_{z}) = (2, 2, 2) for numerical |
| | the x-axis, y-axis, and z- | simulations |
| | axis, respectively, in motion | (Kₓ, K_{y}, K_{z}) = (1, 1, 2) for in vivo |
| | correction | experiments |
| Aₜᵣₐ (m) | The amplitude of the | Aₜᵣₐ = |
| | translation distance in | 0.2, 0.4, 0.6, 0.8, 1.0, 1.2 (mm) for |
| | translation-induced motions | translation-induced motions |
| | (Eqn. 4) | |
| A_{def} | The amplitude of the | A_{def} = |
| | deformation ratio in | 0.02, 0.04, 0.06, 0.08, 0.10, 0.12 for |
| | deformation-induced | deformation-induced motions |
| | motions (Eqn. 5) | |
| Nₚₑᵣ | The number of motion | Nₚₑᵣ = 0.5, 1, 2, 3 for translation- |
| | periods during a 90° rotation | and deformation-induced motions |
| | of the four-arc array (Eqns. | |
| | 4 and 5) | |

| | |
|---|---|
| Dₘ_{_{sub}} | The m_{sub}-th subdomain |
| r′_{c,m}_{_{sub}} (m) | The center of Dₘ_{_{sub}} |
| rₙ_{_{loc}}_{,n}_{ₑₗₑ} (m) | The center of the detection surface of the nₑₗₑ-th transducer element |
| | when the four-arc array is at the n_{loc}-th location |
| ζ(r′, rₙ_{_{loc}}_{,n}_{ₑₗₑ}) (s) | The motion-induced temporal shift of the signals from the source |
| | point at r′ that are detected by the nₑₗₑ-th element when the |
| | transducer array is at the n_{loc}-th location |
| \hat{p}_{ζ}(rₙ_{_{loc}}_{,n}_{ₑₗₑ},t) (Pa) | Detected signals with motion-induced temporal shifts |
| | ζ(r′, rₙ_{_{loc}}_{,n}_{ₑₗₑ}) (Eqn. S36) |
| kₓ(r′_{c,m}_{_{sub}},n_{loc}), | Translation step numbers of the m_{sub}-th subdomain along the x- |
| k_{y}(r′_{c,m}_{_{sub}},n_{loc}), and | axis, y-axis, and z-axis, respectively, when the transducer array is at |
| k_{z}(r′_{c,m}_{_{sub}},n_{loc}) | the n_{loc}-th location |
| k(r′_{c,m}_{_{sub}},n_{loc}) | The translation vector defined as |
| | (kₓ(r′_{c,m}_{_{sub}}, n_{loc}), k_{y}(r′_{c,m}_{_{sub}}, n_{loc}), k_{z}(r′_{c,m}_{_{sub}}, n_{loc})) |
| k | The translation set defined as |
| | {k(r′_{c,m}_{_{sub}}, n_{loc}) ϵ [symbol] ³ \|m_{sub} = 1, 2, . . . , M_{sub}, n_{loc} = 1, 2, . . . , N_{loc}} |
| ζₖ(r′_{c,m}_{_{sub}},rₙ_{_{loc}}_{,n}_{ₑₗₑ}) (s) | Temporal shifts caused by the translations in k (Eqn. S38) |
| ζₖ(r′ₘ,rₙ) (s) | Temporal shifts obtained from τₖ (r′_{c,m}_{_{sub}}, rₙ_{_{loc}}_{,n}_{ₑₗₑ}) |
| | through spatial interpolation |
| \hat{p}_{k,n,l} (Pa) | Detected signals with motion described by k (Eqn. S39) |
| Hₖ | The motion-incorporated system matrix implemented in Eqn. S39 |
| \hat{k}ₓ (r′_{c,m}_{_{sub}},n_{loc}), | Translation step numbers obtained using Eqns. S48, S49, and S50, |
| \hat{k}_{y} (r′_{c,m}_{_{sub}},n_{loc}), | respectively |
| and \hat{k}_{z} (r′_{c,m}_{_{sub}},n_{loc}) | |
| \hat{k} | Reconstructed translation vectors for all subdomains and array |
| | locations |
| z_{tra,n}_{_{loc}} | The translation distance defined in Eqn. 9. |
| α_{def,n}_{_{loc}} | The deformation ratio defined in Eqn. 10. |

### IV. Examples of Systems

The TISMC and hybrid methods described above may be implemented using one or more computing devices (e.g., computing device 1820 in FIG. 18A or computing device 1822 in FIG. 18B). For example, a method may be performed using a computational device such as a server device, a laptop computer, a desktop computer, or the like.

FIG. 18A depicts a block diagram of an example of components of tomographic imaging system 1800 for performing TISMC methods and/or hybrid methods, according to various embodiments. Tomographic imaging system 1800 includes a tomographic imaging device 1810 configured to acquire tomographic images. Some examples of suitable tomographic imaging devices include a PACT system, a CT system, an MRI system, and ultrasound computed tomography (USCT) system. Tomographic imaging device 1810 includes one or more sensor arrays 1812 (e.g., ultrasonic transducer arrays in PACT and USCT, X-ray detection arrays in CT, and RF-coils in MRI). Tomographic imaging system 1800 also includes a computing device 1820 in electrical communication with tomographic imaging device 1810 to receive signals detected by the one or more sensor arrays 1812 such as, e.g., sparsely sampled signals and/or densely sampled signals. In some cases, the computing device 1820 may also send control signals to the tomographic imaging device 1810 to control functions (e.g., sampling) of the tomographic imaging device 1810. Communication between components of tomographic imaging system 1800 may be in wireless and/or wired form. The computing device 1820 may include one or more processors (e.g., a CPU, GPU or computer, analog and/or digital input/output connections, controller boards, etc.) and a non-transitory computer readable medium in electrical communication with the one or more processors.

FIG. 18B depicts components of a computing device 1822, according to embodiments. In one implementation, the computing device 1820 in FIG. 18A includes one or more of the components of the computing device 1822 in FIG. 18B. In various examples, computing device 1822 is in electrical communication with a tomographic imaging device (e.g., tomographic imaging device 1810 in FIG. 18A) to receive signals with tomographic data such as sparsely sampled signals and/or densely sampled signals. In some cases, the computing device 1822 may also send control signals to the tomographic imaging device to control functions of the tomographic imaging device. Communication between components of computing device 1822 may be in wireless and/or wired form.

In FIG. 18B, computing device 1822 includes a bus 1823 coupled to an input/output (I/O) subsystem 1832, one or more processors 1834, one or more communication interfaces 1836, a main memory 1838, a secondary memory 1842, and a power supply 1840. One of more of these components may be in separate housing.

I/O subsystem 1832, includes, or is in communication with, one or more components, which may implement an interface for interacting with human users and/or other computer devices depending upon the application. Certain embodiments disclosed herein may be implemented in program code on computing device 1822 with I/O subsystem 1832 used to receive input program statements and/or data from a human user (e.g., via a graphical user interface (GUI), a keyboard, touchpad, etc.) and to display them back to the user, for example, on a display. The I/O subsystem 1832 may include, e.g., a keyboard, mouse, graphical user interface, touchscreen, or other interfaces for input, and, e.g., an LED or other flat screen display, or other interfaces for output. Other elements of embodiments may be implemented with a computer device like that of computing device 1822 without I/O subsystem 1832. According to various embodiments, the one or more processors 1834 may include a CPU, GPU or computer, analog and/or digital input/output connections, controller boards, etc.

Program code may be stored in non-transitory computer readable media such as secondary memory 1842 or main memory 1838 or both. The one or more processors 1834 may read program code from one or more non-transitory media and execute the code to enable computing device 1822 to accomplish the methods performed by various embodiments described herein, such as TISMC and/or hybrid method. Those skilled in the art will understand that the one or more processors 1834 may accept source code and interpret or compile the source code into machine code that is understandable at the hardware gate level of the one or more processors 1834.

Communication interfaces 1836 may include any suitable components or circuitry used for communication using any suitable communication network (e.g., the Internet, an intranet, a wide-area network (WAN), a local-area network (LAN), a wireless network, a virtual private network (VPN), and/or any other suitable type of communication network). For example, communication interfaces 1836 can include network interface card circuitry, wireless communication circuitry, etc.

In certain embodiments, computing device 1822 may be part of or connected to a controller that is employed to control functions of a tomographic imaging device (e.g., tomographic imaging device 1810 such as controlling data acquisition by one or more sensors (e.g., one or more sensor arrays 1812 in FIG. 18A). The controller will typically include one or more memory devices and one or more processors. The processor may include a CPU or computer, analog and/or digital input/output connections, stepper motor controller boards, etc.

Modifications, additions, or omissions may be made to any of the above-described embodiments without departing from the scope of the disclosure. Any of the embodiments described above may include more, fewer, or other features without departing from the scope of the disclosure. Additionally, the steps of described features may be performed in any suitable order without departing from the scope of the disclosure. Also, one or more features from any embodiment may be combined with one or more features of any other embodiment without departing from the scope of the disclosure. The components of any embodiment may be integrated or separated according to particular needs without departing from the scope of the disclosure.

It should be understood that certain aspects described above can be implemented in the form of logic using computer software in a modular or integrated manner. Based on the disclosure and teachings provided herein, a person of ordinary skill in the art will know and appreciate other ways and/or methods to implement the present invention using hardware and a combination of hardware and software.

Any of the software components or functions described in this application, may be implemented as software code using any suitable computer language and/or computational software such as, for example, Java, C, C#, C++ or Python, Matlab, or other suitable language/computational software, including low level code, including code written for field programmable gate arrays, for example in VHDL; embedded artificial intelligence computing platform, for example in Jetson. The code may include software libraries for functions like data acquisition and control, motion control, image acquisition and display, etc. Some or all of the code may also run on a personal computer, single board computer, embedded controller, microcontroller, digital signal processor, field programmable gate array and/or any combination thereof or any similar computation device and/or logic device(s). The software code may be stored as a series of instructions, or commands on a CRM such as a random-access memory (RAM), a read only memory (ROM), a magnetic media such as a hard-drive or a floppy disk, or an optical media such as a CD-ROM, or solid stage storage such as a solid state hard drive or removable flash memory device or any suitable storage device. Any such CRM may reside on or within a single computational apparatus, and may be present on or within different computational apparatuses within a system or network. Although the foregoing disclosed embodiments have been described in some detail to facilitate understanding, the described embodiments are to be considered illustrative and not limiting. It will be apparent to one of ordinary skill in the art that certain changes and modifications can be practiced within the scope of the appended claims.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

What is claimed is:

1. An intra-image motion correction method, comprising:
(a) dividing an image domain into a plurality of subdomains;
(b) reconstructing a tomographic image;
(c) estimating a motion of a subdomain of the plurality of subdomains along a first axis;
and
(d) reconstructing a first motion-corrected image using the motion of the subdomain along the first axis.

2. The method of claim 1, wherein (c) comprises:
determining a plurality of simulated signals with different subdomain motions along the first axis; and
wherein the motion along the first axis is estimated to be the subdomain motion of the simulated signal that most closely matches a detected signal.

3. The method of claim 1, wherein (c) comprises:
minimizing a difference between a detected signal and a simulated signal based on the motion of the subdomain along the first axis.

4. The method of claim 1,
wherein (c) further comprises:
(c1) estimating a motion of the subdomain along a second axis; and
(c2) estimating a motion of the subdomain along a third axis,
wherein the first motion-corrected image is determined also using the motion of the subdomain along the second axis and the motion of the subdomain along the third axis.

5. The method of claim 1, further comprising:
(e) performing (c) for each sensor array location of a plurality of sensor array locations.

6. The method of claim 5, further comprising:
(f) performing (e) for each subdomain of the plurality of subdomains.

7. The method of claim 4, further comprising:
(f) performing (c) for each subdomain of the plurality of subdomains, wherein the first motion-corrected image is determined using the motions of the subdomains along the first axis, the motions of the subdomains along the second axis, and the motions of the subdomains along the third axis.

8. The method of claim 1, further comprising:
acquiring a detected signal from one or more sensor arrays of a tomographic imaging system.

9. The method of claim 8, wherein the tomographic image at is reconstructed using the detected signal and an initial system matrix.

10. The method of claim 4,
further comprising updating an initial system matrix with the motion of the subdomain along the first axis, the motion of the subdomain along the second axis, and the motion of the subdomain along the third axis to obtain an updated system matrix; and
wherein the first motion-corrected image is reconstructed using a detected signal and the updated system matrix.

11. The method of claim 10, further comprising:
repeating (c), (c1), and (c2) and reconstructing a second motion-corrected image using the motion of the subdomain along the first axis, the motion of the subdomain along the second axis, and the motion of the subdomain along the third axis; and
if a difference between the first motion-corrected image and the second motion-corrected image is greater than a threshold, repeating (c), (c1), and (c2) and reconstructing a third motion-corrected image using the motion of the subdomain along the first axis, the motion of the subdomain along the second axis, and the motion of the subdomain along the third axis.

12. The method of claim 8, wherein each sensor of the one or more sensor arrays is one of an ultrasonic transducer, an X-ray radiation sensor, or a magnetic resonance sensor.

13. The method of claim 8, wherein the one or more sensor arrays comprises an ultrasonic transducer array.

14. The method of claim 8, wherein the one or more sensor arrays comprises a plurality of ultrasonic arc transducer arrays.

15. A system comprising:
a tomographic imaging device comprising one or more sensor arrays;
one or more processors in electrical communication with the tomographic imaging device; and
one or more processor-readable media storing instructions which, when executed by the one or more processors, cause performance of:
(a) dividing image domain into a plurality of subdomains;
(b) reconstructing a tomographic image;
(c) estimating a motion of a subdomain of the plurality of subdomains along a first axis;
and
(d) reconstructing a first motion-corrected image using the motion of the subdomain along the first axis.

16. The system of claim 15, wherein (c) comprises:
determining a plurality of simulated signals with different subdomain motions along the first axis; and
wherein the motion along the first axis is estimated to be the subdomain motion of the simulated signal that most closely matches a detected signal.

17. The system of claim 15, wherein (c) further comprises:
(c1) estimating a motion of the subdomain along a second axis; and
(c2) estimating a motion of the subdomain along a third axis,
wherein the first motion-corrected image is determined also using the motion of the subdomain along the second axis and the motion of the subdomain along the third axis.

18. The system of claim 15, wherein the one or more processor-readable media stores additional instructions which, when executed by the one or more processors, cause performance of:
(e) repeating (c) for each sensor array location of a plurality of sensor array locations.

19. The system of claim 18, wherein the one or more processor-readable media stores additional instructions which, when executed by the one or more processors, cause performance of:
(f) repeating (e) for each subdomain of the plurality of subdomains.

20. The system of claim 17, wherein the one or more processor-readable media stores additional instructions which, when executed by the one or more processors, cause performance of:
updating an initial system matrix with the motion of the subdomain along the first axis, the motion of the subdomain along the second axis, and the motion of the subdomain along the third axis to obtain an updated system matrix; and
wherein the first motion-corrected image is reconstructed using a detected signal and the updated system matrix.

21. The system of claim 20, wherein the one or more processor-readable media stores additional instructions which, when executed by the one or more processors, cause performance of:
repeating (c), (c1), and (c2) and reconstructing a second motion-corrected image using the motion of the subdomain along the first axis, the motion of the subdomain along the second axis, and the motion of the subdomain along the third axis; and
if a difference between the first motion-corrected image and the second motion-corrected image is greater than a threshold, repeating (c), (c1), and (c2) and reconstructing a third motion-corrected image using the motion of the subdomain along the first axis, the motion of the subdomain along the second axis, and the motion of the subdomain along the third axis.

22. The system of claim 15, wherein the one or more sensor arrays comprises at least one of an ultrasonic transducer, an X-ray radiation sensor, or a magnetic resonance sensor.

23. The system of claim 15, wherein the one or more sensor arrays are part of a PACT system, a CT system, or an MRI system.
